(19) United States
(12) Patent Application Publication
(10) Pub. No.: US 2011/0191867 A1
(43) Pub. Date: Aug. 4, 2011

(54) PEPTIDE VACCINE FOR INFLUENZA VIRUS

(75) Inventors: Jari Natunen, Vantaa (FI); Jukka Hiltunen, Helsinki (FI); Ritva Niemela, Helsinki (FI); Jari Helin, Rajamaki (FI); Olli Aitio, Helsinki (FI)

(73) Assignee: GLYKOS FINLAND OY, Helsinki (FI)

(21) Appl. No.: 12/739,796

(22) PCT Filed: Oct. 24, 2008
(86) PCT No.: PCT/FI2008/050598

§ 371 Date: Apr. 26, 2010

(30) Foreign Application Priority Data
Oct. 26, 2007 (FI) PCT/FI2007/050577

(51) Int. Cl. A01K 67/00; C07K 7/64; G01N 33/566; C40B 30/04; G01N 33/53; C12Q 1/70; C40B 30/02

(52) U.S. Cl. 800/8; 530/317; 436/501; 506/9; 435/7.1; 435/5; 506/8

(57) ABSTRACT

The invention provides peptide epitopes for use in the prevention and/or treatment of influenza or for the development of such treatment or vaccine against influenza. The invention also relates to a method for evaluating the potential of a chemical entity, such as an antibody, to bind to a peptide epitope derived from the divalent sialoside binding site of hemagglutinin protein of influenza virus, and to conjugates containing one or more such peptide epitopes. The peptide epitopes of the invention are cyclic peptides comprising a 7-mer peptide derived from H1, H3 or H5 hemagglutinin of influenza virus. The 7-mer peptide has a sequence corresponding to the loop sequence at positions 220-226 of X31-hemagglutinin.

The invention relates to the method for evaluating the potential of a chemical entity, such as an antibody, to bind to a peptide epitope derived from the divalent sialoside binding site of hemagglutinin protein of influenza virus. The invention also provides peptide epitopes for use in the prevention and/or treatment of influenza or for the development of such treatment or vaccine against influenza.

### BACKGROUND OF THE INVENTION

Influenza virus infect the airways of a patient and initially cause general respiratory symptoms, which may result in high morbidity and mortality rates, especially in elderly persons. Thus, good targets for attacking the virus are constantly searched for. The significance of hemagglutinin protein of influenza virus in the pathogenesis of the virus has been known for a relatively long time. Consequently, in the field of vaccine and antibody development an aim has been to develop vaccines against conserved regions of influenza virus hemagglutinins. For example, a patent application of Takara Shuzo (EP0675199) describes antibodies, which recognize the stem region of certain influenza virus subtypes. WO0032228 describes vaccines containing hemagglutinin epitope peptides 91-108, 307-319, 306-324 and for non-caucasian populations peptide 458-467. Lu et al. 2002 describe a conserved site 92-105. Lin and Cannon 2002 described conserved residues Y88, T126, H174, E181, L185 and G219. Hennecke et al. 2000 studied complex of hemagglutinin peptide HA306-318 with T-cell receptor and a HLA-molecule. Some conserved peptide structures have been reported in the primary binding site and a mutation which changes the binding specificity from α6-sialic acids to α3-sialic acids.

There is development of vaccines against different peptides of influenza hemagglutinin on different or partially overlapping sites. An example of different site is the cleavage site of hemagglutinin HA₀ including, e.g., ones developed by Merck and Biondvax. Other development including minor part of somewhat overlapping hemagglutinin peptides including ones developed by Variation biotechnology, e.g, including peptide 1 and peptide 4 described in WO06128294 (Jul. 12, 2006) and Biondvax including peptide HA91 (e.g. WO07066334, Jun. 14, 2007) directed to longer peptides epitopes which are not conformational and conjugated as disclosed in the present invention.

Certain MHCII T-cell epitope peptides directed publications disclosed as prior art for our earlier application PCT/FI2006/050157 were: US 2006002947(D1), WO9859244 (D2), Gelder C et al In Immun. (1998) 10, 211-222 (D3), and D4 J. Virol 1991 65 364-372. Based on the length of the peptides from mouse models and multitude of peptides from which there are varying and partially contradicting results endless number of small peptides could be derived, but the effective definitive peptide epitopes cannot be known.

Targeting virus surface and carbohydrate binding site. The above publications D1-D4 are not targeted to epitopes present only on the surface and on the carbohydrate binding site of the influenza virus. The long sequences are randomly derived from influenza virus and are only partially available for recognition on the surface of virus. It is realized that any immune reaction (cell mediated or antibody mediated) against influenza are useless and misdirected, when not targeted against the surface of the virus proteins, and the result cannot be as good as disclosed in the present invention.

Conserved epitopes. The publications D1-D4 are directed to long peptides specific for single type of influenza virus while present invention is directed to conserved peptide epitopes allowing directing immune reaction to multiple virus strains of major human virus such as H1, H3 or H5 and relevant semiconserved variants thereof. It is realized that misdirected effect against long epitope (as described above) against a single strain is not as useful as the multi strain specific effect according to the invention.

Prior art D1-D4 do not include peptides recognized by antibodies but obligatorily larger MHCII-peptides. The publications D1-D4 describe so called MHCII-receptor mediated, T-cell immune reactions, which are different from the antibody mediated reactions according to the invention. It is obvious to anyone skilled in the art peptide epitopes according to the invention, which are immunogenic and can cause antibody mediated immune reactions in human, cannot be known from the publications directed to different larger peptides and cell mediated immunity. D1-D4 describing large peptides binding to T-cell receptors. The recognition of peptides by T-cell receptors, as indicated in D-publications, would require large peptides, it is indicated in D2 that MHCII binding requires 13-20 amino acid residues (D2 page 1 lines 33-35). All the peptides of D1-D4 are in this range. The present invention is directed especially to conformational epitopes such as cyclic peptides, these are clearly different from the linear background peptides and functions differently e.g. with regard to T-cell receptors, obviously not recognizing cyclic epitopes like linear ones.

Cost and productability. It is further obvious that it is much cheaper, robust and controllable to produce short than long peptides.

Antibody mediated immune responses. The antigenicity of peptide with regard to antibody mediated immune response depend on recognition of the peptides by variable regions of antibodies coded by specific antibody genes (V-, D-, and J-segments) in B-cells (Roitt, Brostoff and Male Immunology fourth edition 1996, or any equivalent general text book). It is obvious that this cannot be determined from T-cells receptor bindings such as indicated in background. This invention revealed that the short peptide epitopes and/or conformational peptides are immunogenic and related to antibody mediated protection against human influenza infection. The present invention indicates antibody mediated immune responses, that are especially useful against influenza.

Analytic use against human natural antibodies. It is further realized that the long peptides suggested in D1-D4 do not reveal usefulness of the present short peptide epitopes in analysis of human antibody mediated immune reactions against the carbohydrate binding site of hemagglutinin. The invention revealed, that there are individual specific differences in immune reactions against the peptides and these correlate to the structures of various influenza virus strains to which the test subject would have been exposed to.

There is development of vaccines against other proteins of influenza such as M2 protein or peptide epitopes are developed by the companies including Merck US (peptides), Acambis (with Flanders Univ.), AlphaVax (with NIH, pandemic), VaxInnate (with Yale Univ.), Dynavax (with support from NIH), Cytos Biotech, CH), GenVec (with NIAID), or Molecular Express, Ligocyte or Globe immune or Biondvax (Israel, Ruth Arnon and colleagues) and known from the background of their publications. M2 also referred as M2e is common (conserved) antigen and ion channel on influenza, it is not accessible on viral surface but targeted on infected cells (assembly of virus) and it does not cure effectively but relieve disease (Science 2006, Kaiser) and NP protein (nucleoprotein of influenza) or peptide epitope are developed e.g. by the companies Biondvax, AlphaVax, GenVec and known from the background of their publications).

It appears that the high affinity bindings caused by the polylactosamine backbone allow effective evolutionary changes between different types of terminally sialylated structures. Currently the influenza strains binding to human are more α6-sialic acid specific, but change may occur quickly. Therefore effective medicines against more “zoonotic” influenzas spreading to human from chicken or possibly from ducks need to be developed. There are examples of outbreaks of “chicken influenza” like the notorious Hong Kong-97 strain, which was luckily stopped by slaughtering all chickens in Hong Kong and thus resulted in only a few human casualties. The major fear of authorities, such as WHO, is the spread of such altered strains avoiding resistance in population based on the previous influenza seasons and leading to global infection, pandemy, of lethal viruses with probable α3-sialic acid binding. A major catastrophe of this type was the Spanish flu in 1918. An outbreak of an easily spreading influenza virus is very difficult to stop. There are currently limitedly effective expensive medicines such as sialidase inhibitors, if effective also against to non-human sialidases, could be of some use and the the present vaccines give only temporary protection.

The present invention is directed to use peptide epitopes and corresponding nucleic acids derived from large sialic acid binding site determined in a previous patent application for analysis analysis and typing of influenza and for therapeutics, especially vaccines and immunogenic medication against influenza viruses, especially human influenza viruses and in another embodiment against influenza viruses of cattle (/or wild animals) including especially pigs, horses, chickens(hens) and ducks. The benefit of the short peptide epitopes is that these direct the immune response precisely to the binding site of influenza and block the spreading of the virus.

In silico screening of ligands for a model structure is disclosed for instance in EP1118619 B1 and WO0181627.

The present invention revealed novel antibody target influenza hemagglutinin peptides, including following properties
1) exposed on the surface of the influenza virus
2) more importantly the peptides are part of carbohydrate binding site of hemagglutinin protein of influenza virus
3) partially conserved and thus useful against multiple strains of influenza
4) cheaper and easier to produce and control
5) not obvious from the longer MHCII-binding peptides, because this interaction requires about 20 meric peptides
6) Based on the very large background of long peptides with varying in vitro data from mainly animal models it is not possible derive effective small epitopes according to the invention. Especially, it is not possible known effective short sequences from large peptides comprising tens or hundreds of small epitopes or the exact lengths of the short epitopes.
7) human natural antibodies can recognize the epitopes, animal data is not relevant with regard to human immune system, especially antibodies
8) associated with antibody mediated immune reactions and the antibodies can effectively block the virus adhesion and the disease
9) useful in assays of human natural antibodies
10) Highly immunogenic variants of the peptides involving current influenza types, especially presently demonstrated variants and analogs thereof.
11) Highly immunogenic variants, which are associated with strong immune reaction in context of vaccination and/or severe influenza infection.
12) The present invention provides especially highly effective conformational presentation involving side chain linked or cyclic conformational structures
13) The present invention effective conjugate structures and polyvalent conjugates for the presentation of the peptides. It is notable that the T-cell directed peptides are especially used as monomeric substances targeting MHC-receptors.
14) Relevant and useful variants and preferred structures among the possible peptides.

It is realized that an antibody mediated immune reaction against such peptide epitope is able to block the binding of the virus and thus stop the infection. It is further realized that it is useful to study antibody mediated immune reactions against the peptides to reveal natural resistance to various types of human infecting influenza viruses.

### SUMMARY OF THE INVENTION

The present invention revealed novel cyclic peptides, which are useful for analysis of immune reactions against human influenza virus. Presence of natural antibodies against the peptides derived from new influenza strains corresponds to recent influenza disease or vaccination was observed from human serum using the novel peptides in a solid phase assay. The invention is in a specific embodiment directed to use of the assay for analysis of human immune reactions correlating to various influenza types and specific new influenza virus strains. The invention is further directed to the use of the peptides for purification and identification of antibodies against the specific peptide epitopes. Furthermore, the present invention reveals useful vaccine molecule conjugates based on the novel conformational, especially cyclic peptides.

Under specific embodiment the present invention includes methods to analyze influenza peptides by genomics methods including PCR. These can be used for the analysis of present target peptide epitopes from new emerging influenza virus strains. Based on sequence comparison of the HA gene from H1, H3 and H5 sequences a series of nucleic acid primers directed to well conserved regions within these genes has been developed. These primers are useful to screen for a wide variety of HA isolates, and allow for screening, treatment, prevention and/or alleviation of influenza caused symptoms by the peptides and peptide antibodies of the present invention. It is further realized that methods to produce nucleic acid such as DNA vaccines are known in the art. The present peptides are in an embodiment produced on proteins by DNA technologies either in vitro or in vivo.

These primers are useful for detecting the presence of influenza A virus HA in a sample, for example a sample derived from an organism suspected of carrying such a virus, and may be used in a reverse-transcription polymerase chain reaction in order to detect the presence of virus in the sample. The primers also encompassing peptide regions of the invention help to identify what antibodies or oligosaccharides of the invention to use.

Specifically, the present invention provides a peptide conjugate according to Formula

[PEP-(y)ₚ-(S)_{q}-(z)ᵣ-]ₙPO

wherein PEP is a peptide epitope;

n is an integer≧1 indicating the number of PEP groups covalently attached to carrier PO; S is a spacer group;

y and z are linking groups so that at least y or z is a linking atom group;

p, q and r are independently 0 or 1 so that at least p or r is 1;

PO is an oligomeric or polymeric carrier structure;

wherein said PEP is a cyclic peptide comprising a 7-mer peptide derived from H1, H3, or H5 hemagglutinin of influenza virus, said peptide having a sequence the location of which in said hemagglutinin corresponds to the loop sequence at positions 220-226 of X31-hemagglutinin.

The present invention also provides cyclic peptide of 7-12 amino acids comprising the sequence RPRVRNI (SEQ ID NO:5), RPRIRNI (SEQ ID NO:6), or RSKVNGQ (SEQ ID NO:7).

### A BRIEF DESCRIPTION OF FIGURES AND SCHEMES

FIG. 1A. Peptide sequence epitopes derived from human H1 viruses.

FIG. 1B. Peptide sequence epitopes derived from human H3 viruses.

FIG. 2. Peptide sequence epitopes derived from human and animal H1, H2, H3, H4, and H5 viruses.

FIG. 3. ELISA binding assay of serum antibodies of test subjects Serum 1B-8B (S1B-S8B) on streptavidin immobilized peptide 1B. Y-axis indicates absorbance units.

FIG. 4. ELISA binding assay of serum antibodies of test subjects Serum 1B-8B (S1B-S8B) on streptavidin immobilized peptide 2B. Y-axis indicates absorbance units.

FIG. 5. ELISA binding assay of serum antibodies of test subjects Serum 1B-8B (S1B-S8B) on streptavidin immobilized peptide 3B. Y-axis indicates absorbance units.

FIG. 6. ELISA binding assay of serum antibodies of test subjects Serum 1B-8B (S1B-S8B) on streptavidin immobilized peptide 4B. Y-axis indicates absorbance units.

FIG. 7. ELISA binding assay of serum antibodies of test subjects Serum 1B-8B (S1B-S8B) on streptavidin immobilized peptide 5B. Y-axis indicates absorbance units.

FIG. 8. Comparison of ELISA binding assays of serum antibodies of test subjects Serum 1B-8B (S1B-S8B) on streptavidin immobilized peptide 1B and peptide 3. Y-axis indicates absorbance units.

FIG. 9. ELISA binding assay of serum antibodies of test subjects Serum 1B-3B and 5B-8B (S1B-S3B, S5B-S8B) on streptavidin immobilized peptide 1C. Y-axis indicates absorbance units.

FIG. 10. ELISA binding assay of serum antibodies of test subjects Serum 1B-3B and 5B-8B (S1B-S3B, S5B-S8B) on streptavidin immobilized peptide 2C. Y-axis indicates absorbance units.

FIG. 11. ELISA binding assay of serum antibodies of test subjects Serum 1B-3B and 5B-8B (S1B-S3B, S5B-S8B) on streptavidin immobilized peptide 3C. Y-axis indicates absorbance units.

FIG. 12. ELISA binding assay of serum antibodies of test subjects Serum 1B-3B and 5B-8B (S1B-S3B, S5B-S8B) on streptavidin immobilized peptide 2B. Y-axis indicates absorbance units.

FIG. 13. ELISA binding assay of serum antibodies of test subjects Serum 1B-3B and 5B-8B (S1B-S3B, S5B-S8B) on streptavidin immobilized peptide 3B. Y-axis indicates absorbance units.

FIG. 14. Comparison of ELISA binding assays of serum antibodies of test subjects Serum 1B-3B and 5B-8B (S1B-S3B, S5B-S8B) on streptavidin immobilized peptide 1C (P 1C) and peptide 2B (P 2B). Y-axis indicates absorbance units.

FIG. 15. Comparison of ELISA binding assays of serum antibodies of test subjects Serum 1B-3B and 5B-8B (S1B-S3B, S5B-S8B) on streptavidin immobilized peptide 3C (P 3C) and peptide 3B (P 3B). Y-axis indicates absorbance units.

### DETAILED DESCRIPTION OF THE INVENTION

This application has priority from WO 2008/049974, the priority application and all prior influenza patent applications of the inventors such as WO 2005/037187 and WO 2006/111616 and other patents and documents mentioned are fully incorporated herein by reference.

The invention reveals novel peptide vaccine compositions, and peptides for analysis and development of antibodies, when the peptides are derived from carbohydrate binding sites of carbohydrate binding proteins (lectins/adhesions) of pathogens, in a preferred embodiment human pathogens such as influenza virus.

The preferred carbohydrate binding sites are carbohydrate binding sites of pathogens comprising large carbohydrate binding sites involving binding to multiple monosaccharide units, more preferably including binding sites for two sialic acid structures. The invention is specifically directed to use of several peptides derived from carbohydrate binding site(s) of a pathogen surface protein, preferably from different parts of the carbohydrate binding site, more preferably from two different sialic acid epitope binding sites or one sialic acid binding site and conserved/semiconserved carbohydrate binding site bridging the sialic acid binding sites.

The invention reveals that conserved or semiconserved amino acid residues form reasonably conserved peptide epitopes at the binding sites of sialylated glycans, preferably binding sites disclosed in the invention. The preferred peptides are derived from the hemagglutinin protein of human influenza protein. It is realized that these epitopes can be used for development of antibodies and vaccines.

The useful antigenic peptides disclosed in the invention are available on the surface of the pathogen, preferably on viral surface.

The peptides which are 1) derived from the carbohydrate binding site (or in a separate embodiment more generally from a conserved binding site of low molecular weight ligand) and which are 2) present on the surface of a pathogen are referred here as “antigen peptides”.

Peptide 1, Peptide 2 and Peptide 3

The invention revealed specific linear amino acid sequences from the large carbohydrate binding site of influenza A viruses, which are useful for studies of binding of antibodies, selection of antibodies and immunizations. Furthermore it was revealed that the regions can be effectively analysed from nucleic acid of influenza virus by PCR-methods. In a preferred embodiment the analysis of nucleic acids is used as a first test for defining a new peptide. More preferably, the peptide 1 is conjugated from a residue corresponding to cysteine 97 or peptide 2 is conjugated from a residue corresponding to cysteine 139 as defined by the amino acid sequence of X31-hemagglutinin.

Peptide 1 comprises a hepta peptide epitope core starting from amino acid residue position corresponding to the position 91 of influenza H3 X31 sequence and ending at cysteine residue 99.

Examples of peptide epitope core from H3 includes, SKAFSNC in X31, and in recent/current viruses especially SKAYSNC and more rare SKADSNC, and STAYSNC, examples of H1 peptide epitope cores includes NSENGTC, NPENGT, and NSENGIC. It is realized that the Other influenza virus A hemagglutinins can be aligned with X31 sequence as shown in Figures and Tables.

Peptide 2 comprises a hepta peptide epitope core starting from amino acid residue position corresponding to the position 136 of influenza H3 X31 sequence and ending residue 141 including at cysteine residue 139. Examples of peptide 2 epitope core from H3 includes, GSNACKR in X31, and in recent/current viruses especially GSYACKR and more rare recent GSSACKR and even more recent TSSACKR(R) (e.g. (A/Nagasaki/N01/2005) or, TSSACIR(R) (e.g. A/USA/AF1083/2007) or SSSACKR(R) (e.g. A/Wisconsin/67/2005) examples of H1 peptide epitope cores includes (G)VTAACSH, and (G)VTASCSH, e.g (N-terminal G is preferred additional residue) and more recently (G)VSASCSH (A/Thailand/CU75/2006).

Peptide 3 comprises a hepta peptide epitope core starting from amino acid residue position corresponding to the position 220 of influenza H3 X31 sequence and ending residue 226. Examples of peptide 3 epitope core from H3 includes, RPWVRGL in X31, and in recent/current viruses especially RPRVR(D/N)(V/I/X)(P),

where in the last residue is V or I or other residue X and a preferred C-terminal additional residue is P, which is preferred because it affect the conformation of the peptide, in a preferred embodiment or RPRVRNI(P), as in new virus (A/Nagasaki/N01/2005) and RPRIRNI(P) (e.g. A/Wisconsin/67/2005).

Examples of H1 peptide 3 epitope cores includes RPKVRDQ common H1, Table 10. The invention revealed by antibody binding studies that cyclic from comprising the core heptapeptide are especially effective. The preferred peptides 3 further includes homologous H5 virus peptides such as RPKVNGQ and similar as defined in the invention.

In the preferred cyclic form both first additional residues from N-terminus and C-terminus are replaced by cysteine or cysteine analogous residue forming disulfide bridge or analogous structure. The sequence may further comprise one or more, preferably 1-6, more preferably 1-4 additional residues X₄X₃X₂ and/or Y₂Y₃Y₄ or a sequence of up to 100 amino acid residues, preferably up to 30 residues derived from the influenza hemagglutinin. Cysteine at N-terminus or X₁ replaces the amino acid residue corresponding to the position 219 in X31, X₂ preferably is an amino acid similar or same as in position 218 in a influenza hemagglutinin, preferably in the hemagglutinin from which the cyclic epitope is derived from the same hemagglutinin as the cyclic structure.

The invention is further directed to truncated epitopes of the peptides so that one or two N-terminal and/C-terminal residues are omitted, the preferred peptides comprise preferably a short peptide epitopes of three or four amino acid residues in the middle of sequences, consensus of this sequence can be used for recognition of specific peptide type according to the invention. In a preferred embodiment the peptide epitope comprise additional amino acid residues according to the invention, such 1-4 amino acid, more preferably 1-3 or even more preferably 1-2 amino acid residues residues on N-terminal and/or C-terminal side of the peptide epitope core. The additional amino acid residues are included with provision, that when the peptide is used as linear peptide without conformational presentation and/or conjugation according to the invention the length of the peptide is preferably 12 amino acid residues or less and as described for the preferred short peptides according to the invention

These additional amino acid residue, when derived from consecutive amino acid residues of influenza virus have function in supporting the conformation of the preferred short peptide epitopes or as spacers or part of an immuno activating structure. The peptides may further comprise additional amino acid sequence from influenza virus, especially when the peptides are preferred conformational peptides according to the invention.

General Presentation of the Core Peptide with Additional Residues

The general sequence of the short peptide epitopes according to the invention are

X₄X₃X₂X₁C₁C₂C₃C₄C₄C₅C₆C₇Y₁Y₂Y₃Y₄

wherein C₁C₂C₃C₄C₄C₅C₆C₇ are core peptide epitope core amino acid residues defined as consensus sequence for specific peptide 1-3 type in the invention, so that the characteristic short (or very short) peptide epitope may be truncated peptide may be truncated by removing one or two of C₁C₂ and C₆C₇ or even more to obtain shorter peptide core epitope of 3- to 6 amino acid residues, which can be used for the recognition of the peptides according to the invention.

X₄X₃X₂X₁ and Y₁Y₂Y₃Y₄ are N-terminal or C-terminal additional amino acid residues, respectively so that the length of the peptide is preferably 12 or less, additional aminoaacid residues and their variants can be added from previous (prey. pre) and post specifications of the peptides according to the invention including ones in Figures/Tables.

The consensus formulas of present invention can be transferred to this type of formula by replacing residues of C₁C₂C₃C₄C₄C₅C₆C₇ by the specific amino acid residues and their variants.

Length of Preferred Epitopes of Antigen Peptides

“Short Epitopes” of About 5-13 Amino Acid Residues

“Very short epitopes” of 3-8 or about 5 amino acid residues. Prior art has studied long peptides covering usually 10-20 amino acid residues. The present invention is directed to peptide epitopes exposed on the viral surface. The epitopes are selected to direct immune reactions to conserved linear epitopes. The epitopes are relatively short about 5 amino acid residues long, preferably 3 to 8 amino acid residues, more preferably 4 to 7 amino acid residues, most preferably 5 to 6 amino acid residues long. The invention reveals that a very short epitope can be enough for recognition by antibodies. The present invention also reveals specific novel conformational peptide epitopes, wherein the most important peptide part is only a few even 3 amino acid residues. The invention is further directed to the peptides of specific regions (A, B and C) in the large sialoside binding site of influenza virus hemagglutinin, wherein the short peptides comprise specific very short epitopes of at least three amino acid residues, preferably 3 amino acid residues of peptides 1-3. It is realized that the peptides mutate but these can be recognized as peptides according to the invention from the specific structures of very short peptide epitopes.

Preferred Short Peptides of 5-13 or 5-12 Amino Acid residue

In a preferred embodiment the invention is directed to specific peptides which have useful conformation for recognition by antibodies comprising at least 5 amino acid residues, more preferably at least 6 amino acid residues. The peptides do not have typical length of over 13 amino acid residues for recognition as T-cell peptides (regular influenza peptide vaccines comprise 16 or 20 meric or larger hemagglutinin peptides). The preferred length of the peptides are thus 5-13, more preferably 5-12 or 6-12 amino acid residues. The preferred optimal influenza surface peptides have lengths of 6-11, more preferably 6-10, or even more preferably 7-10 amino acid residues to include effective binding and conformation epitopes but omitting redundant residues.

The invention is in a preferred embodiment directed to conformational epitopes presented on hemagglutinin surface in the large sialoside binding site, as it is realized that antibodies against these cause effective blocking of the infection. The invention is directed to the use for immunizations of preferably conformational epitopes which can elicit immune responses by leukocytes, especially lymphocytes and most preferably B-cells.

Additional residues to improve presentation. The very short peptide epitope of about 3-8 amino acid residues long sequence preferred amino acid epitopes may be further linked to assisting structures. The preferred assisting structures includes amino acid residues elongating the short epitope by residues giving additional binding strength and/or improving the natural type presentation of the short epitopes. Additional residues may be included at amino terminal and/or carboxy terminal side of the short epitopes. Preferably there are 1-7, additional residues on either or 1-3 both side of the very short epitopes, more preferably 2-4 additional residues. The additional residues are represented, e.g., as prev/pre and past residues or as first residues of following post peptide.

Conformational structures. The preferred short epitopes and/additional residues may further include conformational structures to improve the three dimensional presentation of the short epitope. The preferred conformational structures includes
A) conformational conjugation structures,such as a chemical linker structure improving the conformation of the peptides
B) single amino acid residue presentation improvement, which preferably includes replacement of non-accessible single residue, with a non-affecting structure such as linkage to a carrier or replacement by alanine or glycine residue.

The conformational structures include natural 3D analogues of the epitopes on the viral surfaces:
1) disulfide bridge mimicking structures, which may include natural disulfide bridges or chemical linkages linking cysteine residues to carrier
2) bridging structures including bridging structures forming a loop for natural type representation bridging between two peptide epitopes

The preferred peptide epitopes according to the invention comprise

a) a conformational peptide epitope comprising at least one cysteine residue or cysteine analogous amino acid residue conjugated from the side chain, and the peptide epitope comprises less than 100 amino acid residues, preferably less than 30 amino acid residues present in a natural influenza virus peptide and/or

b) the peptide epitope is a short peptide epitope comprising 3 to 12 amino acid residues, preferably comprising less than 12 amino acid residues, more preferably less than 11 amino acid residue.

In a preferred embodiment the peptide epitope is a conformational peptide epitope and a short peptide epitope.

Preferred conformational peptide epitopes include:

i) peptide 1 or peptide 2, which is conjugated from a cysteine or cysteine analogous residue side chain of the peptide epitope or

ii) peptide 3, which is in a cyclic form via a bridge formed by adding cysteine residues or cysteine analogous residues to the peptide sequence to form a loop comprising conformation similar to peptide loop on the surface of hemagglutinin protein.

More preferably, the peptide 1 is conjugated from a residue corresponding to cysteine 97 or

peptide 2 is conjugated from a residue corresponding to cysteine 139 as defined by the amino acid sequence of X31-hemagglutinin.

The preferred peptide 3 epitope comprises a cyclic or loop conformation of peptide 3, preferably a peptide of seven amino acid residue is cyclized by adding cysteine residues or cysteine analogous residues to N- and C-terminus of the peptides and forming a disulfide bridge or disulfide bridge analogous structure. Preferably, the cyclic or loop conformation has conformation similar to the conformation of peptide 3 on the surface of influenza virus hemagglutinin.

Conjugates

It is realized that it is useful and preferred to represent the peptide epitopes according to the invention in a assay and/or binding method as a conjugated form. The background describes passive absorption of peptides but the present invention reveals very effective and robust assay, when the peptides are specifically conjugated covalently or by strong non-covalent linkage. The invention is further directed to specifically conjugated or covalently conjugated conformational epitopes represented for the immune system. In a preferred embodiment the invention is directed to conjugated structure, wherein the peptide is conjugated from the N-terminal or C-terminal end of the peptide sequence. In another preferred embodiment the peptide is conjugated only from N-terminal end, the invention revealed that such peptides can be effectively recognized by antibodies. In yet another preferred embodiment the peptide is conjugated from both N-terminal and C-terminal and to solid phase or soluble carrier.

In a preferred embodiment the peptide/peptide epitope according to the invention is separated from the carrier or solid phase by a linking atom group and/or linking atom group and a spacer. It is realized that the carrier or solid phase may affect the conformation of the conformational peptide. It is further realized that too long spacer structure would restrict the possibilities for the effective recognition of the peptides.

The invention is especially directed to representation of the conformational cyclic peptide with a flexible and inert spacer comprising a chain of one to five flexible atom structures connected with multiple single bonds such methylene (—CH₂—) groups, ether/oxy groups (—O—) or secondary amine group so that the spacer comprises at least one methylene group (—CH₂—) and more preferably at least two methylene, and even more preferably at least three methylenmethylene groups, the spacer comprise preferably not more than two and more preferably one or no rigid atom structures such as a double bond between carbon residues or an amide bond. In a preferred embodiment the spacer is an aminoalkanoic acid, preferably 2-8 carbon aminoalkanoic acid, more preferably 3-7 carbon aminoalkanoic acid and even more preferably 4-6 amino alkanoic acid such as aminohexanoic acid (amino caproic acid).

When the non-covalent linking structure is biotin, the biotin residue is considered totally being part of the linking structure, and the present invention is preferably directed to conjugating the biotin to the peptide by a flexible spacer, in a preferred embodiment the spacer is alkyl-chain in a preferred aminoalkanoic acid.

The invention is further directed to polyvalent presentation of the peptides according to the invention preferably conformational peptides according to the invention. It is realized that polyvalent presentation is especially useful when the peptides are aimed for inducing lymphocyte, especially B-cell meditated immune reactions/responses. especially for antibody production.

Polyvalent Conjugates

The present invention is further directed to influenza binding directed analysis or therapeutic substance according to the formula PO

[PEP-(y)ₚ-(S)_{q}-(z)ᵣ-]ₙPO (SP1)

wherein PO is an oligomeric or polymeric carrier structure, PEP is the peptide epitope sequence according to the invention, PO is preferably selected from the group: a)solid phases, b) immunogenic and or oligomeric or polymeric carrier such as multiple antigen presenting (MAP) constructs, proteins such as KLH (keyhole limpet hemocyanin oligosaccharide or polysaccharide structure,

n is an integer≧1 indicating the number of PEP groups covalently attached to the carrier PO, S is a spacer group, p, q and r are each 0 or 1, whereby at least one of p and r is different from 0, y and z are linking groups, at least one of y and z being a linking atom group also referred as “chemoselective ligation group”, in a preferred embodiment comprising at least one an O-hydroxylamine residue —O—NH— or —O—NH═, with the nitrogen atom being linked to the OS and/or PO structure, respectively, and the other y and z, if present, is a chemoselective ligation group, with the proviso that when n is 1, the carrier structure is a monovalent immunogenic carrier. In a preferred embodiment linking atom group z is biotin or equivalent ligand capable of specific strong non-covalent interaction.

In a preferred embodiment the conjugate comprises additional y2 or y2 and y3 groups forming additional linkages from N- or C-terminus or middle cysteine position to PEP to enhance the presentation of the conformational peptide group.

Chemoselective Ligation Groups

The chemoselective ligation group y and/or z is a chemical group allowing coupling of the PEP-group to a spacer group or a PEP−(y)ₚ−(S)_{q}−(z)ᵣ− group to the PO carrier, specifically without using protecting groups or catalytic or activator reagents in the coupling reaction. According to the invention, at least one of these groups y and z is a O-hydroxylamine residue —O—NH— or —O—N═. Examples of other chemoselective ligation groups which may be present include the hydrazino group —N—NH— or —N—NR₁—, the ester group —C(═O)—O—, the keto group —C(═O)—, the amide group —C(═O)—NH—, —O—, —S—, —NH—, —NR₁—, etc., wherein R₁ is H or a lower alkyl group, preferably containing up to 6 carbon atoms, etc. A preferred chemoselective ligation group is the ester group —C(═O)—O— formed with a hydroxy group, and the amide group —C(═O)—NH— formed with an amine group on the PO or Bio group, respectively. In a preferred embodiment, y is an O-hydroxylamine residue and z is an ester linkage. Preferably p, q, and r are 1. If q is 0, then preferably one of p and r is 0.

Preferred polysaccharide or oligosaccharide backbone (PO) structures include glycosaminoglycans such as chondroitin, chondroitin sulphate, dermantan sulphate, poly-N-acetylactosamine or keratan sulphate, hyaluronic acid, heparin, and heparin precursors including N-acetylheparosan and heparan sulphate; chitin, chitosan, starch and starch or glycogen fractions and immunoactivating glucose polysaccharides (e.g pullulan type polysaccharides or beta-glucans such as available from yeast) or mannose (such as mannans) polysaccharides and derivatives thereof. A preferred backbone structure is a cyclodextrin. Useful starch fractions includes amylose and amylopectin fractions. The invention is specifically directed to use of water soluble forms of the backbone structures such as very low molecular weight chitosan polysaccharide mixture or c and on the other hand non-soluble or less soluble large polysaccharide especially for large polyvalent presentation especially for vaccines and immunizations.

Preferred spacer structure includes ones described for hydrophilic linker above, aminooxyacetic acid. According to an embodiment of the invention the spacer group, when present, is preferably selected from a straight or branched alkylene group with 1 to 10, preferably 1 to 6 carbon atoms, or a straight or branched alkenylene or alkynylene group with 2 to 10, or 2 to 6 carbon atoms. Preferably such group is a methylene or ethylene group. In the spacer group one or more of the chain members can be replaced by —NH—, —O—, —S—, —S—S—, ═N—O—, an amide group —C(O)—NH— or —NH—C(O)—, an ester group —C(O)O— or —O—C(O)—, or —CHR₂, where R₂ is an alkyl or alkoxy group of 1 to 6, preferably 1 to 3 carbon atoms, or —COOH. Preferably a group replacing a chain member is —NH—, —O—, an amide or an ester group.

Hydrophilic Spacer

The invention shows that reducing a monosaccharide residue belonging to the binding epitope may partially modify the binding. It was further realized that a reduced monosaccharide can be used as a hydrophilic spacer to link a receptor epitope and a polyvalent presentation structure. According to the invention it is preferred to link the peptide PEP via a hydrophilic spacer to a polyvalent or multivalent carrier molecule to form a polyvalent or oligovalent/multivalent structure. All polyvalent (comprising more than 10 peptide residues, preferably more than 100 and for vaccination even more that 1000 up to 100 000 or million or 10 000 000 million or more in large polyvalent conjugates) and oligovalent/multivalent structures (comprising 2-10 peptide residues) are referred here as polyvalent structures, though depending on the application oligovalent/multivalent constructs can be more preferred than larger polyvalent structures or vice versa. The hydrophilic spacer group comprises preferably at least one hydroxyl group or alkoxy/ether group. More preferably the spacer comprises at least two hydroxyl groups and most preferably the spacer comprises at least three hydroxyl groups.

According to the invention it is preferred to use polyvalent conjugates in which the hydrophilic spacer group linking the peptide sequences to polyvalent presentation structure is preferably a flexible chain comprising one or several —CHOH— groups and/or an amide side chain such as an acetamido —NHCOCH₃ or an alkylamido. The hydroxyl groups and/or the acetamido group also protects the spacer from enzymatic hydrolysis in vivo. The term flexible means that the spacer comprises flexible bonds and do not form a ring structure without flexibility. A reduced monosaccharide residues such as ones formed by reductive amination in the present invention are examples of flexible hydrophilic spacers. The flexible hydrophilic spacer is optimal for avoiding non-specific binding of neoglycolipid or polyvalent conjugates. This is essential optimal activity in bioassays and for bioactivity of pharmaceuticals or functional foods, for example.

A general formula for a conjugate with a flexible hydrophilic linker has the following Formula HL:

[PEP-(X)ₙ-L₁-CH(H/{CH₁₋₂OH}ₚ₁)—{CH₁OH}ₚ₂—{CH(NH—R)}ₚ₃—{CH₁OH}ₚ₄-L₂]ₘ-Z

wherein L₁ and L₂ are linking groups comprising independently oxygen, nitrogen, sulphur or carbon linkage atom or two linking atoms of the group forming linkages such as —O—, —S—, —CH₂—, —NH—, —N(COCH3)-, amide groups —CO—NH— or —NH—CO— or —N═N— (hydrazine derivative) or hydroxylamine —O—NH— and —NH—O—. L1 is linkage from hydrophilic spacer to additional spacer X or when n=0, L1 links directly from N- or C-terminus or middle cysteine position to PEP.

p1, p2, p3, and p4 are independently integers from 0-7, with the proviso that at least one of p1, p2, p3, and p4 is at least 1. CH₁₋₂0H in the branching term {CH₁₋₂OH}ₚ₁ means that chain terminating group is CH₂OH and when the p1 is more than 1 there is secondary alcohol groups —CHOH— linking the terminating group to the rest of the spacer. R is preferably acetyl group (—COCH₃) or R is an alternative linkage to Z and then L₂ is one or two atom chain terminating group, in another embodiment R is an analog forming group comprising C₁₋₄ acyl group (preferably hydrophilic such as hydroxy alkyl) comprising amido structure or H or C₁₋₄ alkyl forming an amine. And m>1 and Z is polyvalent carrier. PEP is peptide according to the invention, X is additional spacer such as spacer S in formula PO.

Preferred Novel Peptides and Peptide Compositions

The invention is especially directed to peptide 3 and its cyclic and/or elongated variants, more preferably cyclic and optionally elongated variants and combinations thereof.

The invention is further directed to peptides 1-3 and short and/or conformational forms thereof as antigenic peptide or peptide composition comprising at least one peptide, preferably peptide 2 or peptide 3.

The peptides 2 and 3 were observed to be targets of especially effective immune responses, specifically antibody responses. The preferred peptide 2 and 3 three includes H1, H3, and H5 peptides, more preferably H1 and H3, and conformational and/or short peptide, more preferably the peptides are from human infecting influenza virus variants.

In another preferred embodiment, the antigenic peptide composition comprises at least two peptides selected from the group peptide 1, peptide 2 and peptide 3, and in another embodiment all three peptides peptide 1, peptide 2 and peptide 3, and in a preferred embodiment both of the highly immunogenic peptides 2 and 3.

Combinations of Cyclic and Linear Forms of Peptide 3

In a preferred embodiment the invention is directed to preferred methods to analyze antibody specificity using both cyclic and linear peptide variant of peptide 3, and optionally further an elongated variant of cyclic and/or linear peptide. The invention is especially directed measuring patients or test animal serum binding to both linear and cyclic form of the peptides, preferably to reveal effective immunization against the three dimensional structure or linear structure of the the peptide by a vaccine or in other embodiment by natural virus.

The invention is further directed to a method to purify or select an antibody binding to peptide 3 using both cyclic and linear peptide, the method involving step of isolating antibodies binding effectively to the cyclic peptide but not to linear peptide, eg. by affinity chromatography, or magnetic beads.

Consensus Sequences of Peptides 3

The preferred current H1, H3 and H5 hemagglutinins comprise consensus sequences

(C)ₙRX₁X₂VX₃, wherein

X₁ is P or S;

X₂ is R or K, it realized that these are similar kationic residues;

X₃ is R or N,

(C) is optional cysteine of the cyclic peptide and n is 0 or 1 indicating its presence or absence.

The invention revealed that the preferred hemagglutinin types have pair wise homologies. In a preferred embodiment the invention is directed to H1 and H5 hemagglutinin peptides 3 as a group and H3 as a separate group. In another preferred embodiment the invention is directed to H1 and H3 as a group and the avian type influenza H5 as a separate group.

Preferred Consensus Sequence for H1 and H5 Peptides

It is realized there is very substantial homology between referred H1 and H5 peptides With consensus sequence according to the Formula

(C)ₙRX₁KVX₃, wherein

X₁ is P or S,

X₃ is R or N,

(C) is optional cysteine of the cyclic peptide and n is 0 or 1 indicating its presence or absence, or

In a preferred embodiment a shorter consensus:

according to the Formula

(C)ₙRX₁KV, wherein

X₁ is P or S,

(C) is optional cysteine of the cyclic peptide and n is 0 or 1 indicating its presence or absence

and in yet another embodiment a long consensus sequence

(C)ₙRX₁KVX₃X₄Q(C)ₙ, wherein

X₁ is P or S,

X₃ is R or N,

X₄ is G or D,

(C) is optional cysteine of the cyclic peptide and n is 0 or 1 indicating its presence or absence.

Preferred Consensus Sequence for H1 and H3 Peptides

It is realized there is substantial homology between referred H1 and H3 peptides With consensus sequence according to the Formula

(C)ₙRPX₂VX₃, wherein

X₂ is R or K, it realized that these are similar cationic residues;

X₃ is R or N.

(C) is optional cysteine of the cyclic peptide and n is 0 or 1 indicating its presence or absence.

Preferred Consensus Sequence for H3 and H5 Peptides

It is realized there is substantial homology between referred H3 and H5 peptides With consensus sequence according to the Formula

(C)ₙRX₁X₂VX₃, wherein

X₁ is P or S;

X₂ is R or K, it realized that these are similar kationic residues;

X₃ is R or N.

(C) is optional cysteine of the cyclic peptide and n is 0 or 1 indicating its presence or absence.

Use of the Combinations Comprising Linear and Cyclic Peptide 3

The invention revealed that there are simultaneous immune reactions against linear and cyclic peptide 3, therefore in a preferred embodiment the the vaccine against the cyclic peptide is combined with immunization by conjugate of linear (non-cyclic peptide) according to the Formula of claim 1. It is further realized that use of linear and cyclic peptide together in analysis of antibodies from human or test animal derived samples is preferred and useful method to assess the presence of really conformation specific immune response against the peptides according to the invention.

Further embodiment of the invention is the use of combination of cyclic peptide 3 with linear peptide 2 or linear peptide 1, preferably with linear peptide 2.

Conjugate Structures

It is realized that the peptide conjugates of claim 1 does not include as spacer of carrier a longer peptide sequence continuous to the same or other influenza hemagglutinin, unless cysteine bridge is included in the sequence. It is further realized that peptide 3 is conjugated from either N-terminus or C-terminus and the other terminus may comprise a few additional amino acid residues of the consecutive hemagglutinin sequence according to the invention and/or another spacer to PO or direct linkage to PO including immunomodulatory peptides or molecules.

In a preferred embodiment the conjugate comprises the cyclic peptide conjugated from N- or C terminus preferably both to a protein or peptide structure of a viral like particle influenza vaccine known in the art, wherein the protein is not influenza hemagglutinin. More preferably the cyclic peptide is linked to the protein or peptide structure by a few, such as 1-10 spacer amino acid residues, such as flexible glycine residues. It is realized that such viral like particle or virus vaccines can be produced by genetic engineering and recombinant nucleic acid technologies

Methods for Binding and Selection of Molecules, Especially Antibodies Against the Peptides

Influenza Antibody Target Peptides on Carbohydrate Binding Site

The invention revealed specific peptides which are located on surface of influenza virus divalent sialoside binding site. The peptides can be recognized by antibodies, which then can block the binding to the large binding site also referred as divalent sialoside binding site on the surface of influenza virus. The peptides are thus targets for antibody recognition methods and antibody selection methods based on the specific recognition of the peptides by antibodies.

The antibody recognition method measures of binding of one or more antibody to the peptides. The antibody selection method further involves selection of the binding antibodies, which have desired binding affinity.

Antibody Fragments, Peptides and Equivalent Binding Reagents

It is further realized that multiple other binding reagents equivalent of antibodies or modulator molecules can be selected similarly as antibodies. In a preferred embodiment the other binding reagents are proteins with varying structures like antibodies, antibody fragments or peptides or part of repetitive oligomeric or polymeric structure resembling peptides such as peptide mimetics, which are well known in the art, or nucleic acid derived binding molecules with repetitive structure such as aptamers or a molecule derived from a molecular library comprising molecules large enough for binding.

It is realized that production of a molecular library for screening of binding reagents against the peptides according to the invention is a routine process known for skilled person and when the molecular library is large enough the finding of suitable other binding reagents is feasible.

It is further realized that the binding reagents have inherently common chemical structures corresponding to the three dimensional structures represented by the peptides on the influenza hemagglutinin surfaces. The peptides according to the invention are naturally located on protein surface and thus comprise at least one amino acid residue comprising polar side chain, more preferably at least two, even more preferably at least three polar side chains. The preferred binding structures recognizing the peptide by hydrogen bond or ionic interactions further includes at least one, more preferably at least two and most preferably at least three polar functional group such as a hydroxyl group, carboxy group including keto group, carboxylic acid group, or aldehyde group, amine group or oxygen linked to phosphorus or sulphur atom such as in sulphate, sulfonyl or phosphate structures or polar halogen atoms such as flouro-, chloro- or bromo-halogens, more preferably fluoro or chloro-linked to carbon atoms. The invention is directed to the recognition of hemagglutinin peptides by a reagent comprising at least the same amount of polar structures as represented by the desired target hemagglutinin peptide. The invention is further directed to the recognitions of non-polar structures included in the peptide structures by non-polar structures such as non-polar amino acids or amino acid mimetics on the binding reagents.

Antibody Selection Methods

It is realized that antibodies can be selected in numerous ways involving the step of binding of antibody to the peptide and selection of antibodies binding to the target peptides with desired binding affinity. The preferred binding and/or selection methods include contacting the peptide with a library or multitude of proteins being antibody production involved proteins such as antibodies or molecules representing peptides antibodies. Preferably, the contacting occurs on the surface of genetic entities, such as cells bacteria, or phages, viruses or alike, capable of representing a variant of antibody production involved proteins. In a preferred embodiment genetic entities include immune cells such as leukocytes, preferably lymphocytes, representing antibodies or phages or bacteria representing antibodies or in another embodiment preferred genetic entities include immune cells such as leukocytes, preferably lymphocytes, representing T-cell receptors and/or HLA antigens

The invention is especially directed to the representation of the peptides in libraries of antibodies or antibody fragments for activation of immune cells by the peptides, or in phage display libraries to observe binding of strongly binding antibodies.

The peptides were selected based on the location on the virus surface. It is realized that immunization or selection of antibodies with different longer peptides would produce immune reactions against structures outside of the binding site of the antibodies.

The methods of binding to influenza virus peptides according to the invention, wherein the method is used for selection of chemical entities, preferably antibodies, preferably from a library of the entities and the selection is performed in vivo, ex vivo or in vitro and optionally the detection is observing the result of the selection.

The preferred method involves specific conjugation of the peptide to matrix by a covalent bond or strong non-covalent interaction.

The covalent bond is preferably formed from sulphur atom of a cysteine residue, preferably to maleimide or analogous structure or to a sulphur of cysteine in the matrix or the strong non-covalent interaction is binding of a ligand to a protein, preferably biotin binding to an avidin protein and preferably the peptide is biotinylated.

The binding and/or selection method is in a preferred embodiment an in vitro immunoassay or in vitro selection of an antibody library such as phage display antibody library, preferably involving extensive washing.

In another embodiment the method is an ex vivo or in vivo immunization method, preferably involving activation of immune cells, more preferably lymphocytes, most preferably B-cells.

Search and Evaluation of Potentially Autoimmunogenetic Peptides Form Databases and Protein Conformations

In a preferred embodiment the binding and/or selection method involves a step of searching any of the peptide epitopes 1-3 of an hemagglutinin from database comprising human genome coded peptide sequences and selection of peptides, which are not expected to cause immune reaction against a human (or animal) subject.

In the preferred search method, when similar peptide sequence(s) is (are) found from human (or animal) genome sequence, these will be evaluated with regard to

i) availability for human (animal) immune system with regard to presence of the peptide sequence on surface of a protein and/or on a cell surface protein and preferably selecting peptides which are not available for human (animal) immune system and/or

ii) conformation of the peptide in a human (or animal) protein being similar to conformation of the peptide on the hemagglutinin surface and preferably selecting peptides which do not have similar conformations on human proteins.

Recognition by immune system. The peptides are recognizable by the immune system of the patient and can induce immune reaction against the peptides. The immune reaction such as an antibody reaction and/or cell mediated immune reaction can recognize the peptide epitope on the surface of the virus and diminish or reduces its activity in causing disease. In a preferred embodiment the invention is specifically directed to peptides recognized by antibodies of a patient and development of such peptides to vaccines.

Preferred immune recognition by relevant species such as human and/or pandemic animal species. It is realized that most of the prior art has studied the immunoreactivity of various, in general long, peptide epitopes with regard to species used for immunological experiments such as mice, rats, rabbits or guinea pigs. It is realized that studies with regard to these immune systems is not relevant with regard to the human disease and there is multitude of results supporting this fact. The results have been very varying and does not reveal useful short epitopes with regard to human immune system.

The present invention is directed to analysis of the effect of the antigen peptides in animal species from which influenza infection is known to effectively spread to humans (see U.S. patent application No. 20050002954). Preferred animal species are avian species and/or pig. The preferred avian species includes poultry animals such as chicken and ducks, and wild bird species such as ducks, swans and other migratory water birds spreading influenza virus.

The present invention revealed that the short peptide epitopes are useful against viruses spreading from the relevant species to human patients. It was realized that the epitopes are recognizable on the surfaces of viruses and antibodies binding to peptides would block the carbohydrate binding sites of the viruses.

Screening of antibodies. The invention is directed to screening methods to reveal natural antibodies binding to peptides, preferably peptides derived from carbohydrate binding sites of human pathogens especially carbohydrate binding sites of pathogens comprising large carbohydrate binding sites involving binding to multiple monosaccharide units, more preferably including binding sites for two sialic acid structures. Preferably the invention is directed to screening of human natural antibody sequences against peptides derived from viruses or bacteria, more preferably against carbohydrate binding sites, or binding site peptides, of influenza viruses.

It is realized that antibodies may be screened by affinity methods involving binding of antibodies to the peptide epitopes. The peptide epitopes may be conjugated to solid phase for the screening, preferably for screening of human antibodies. In a preferred embodiment the peptides are screened from blood, blood cells or blood derivative such as plasma or serum of a patient. In another embodiment the antibodies are screened from a phage display library derived from blood cells of a patient or several patients or normal subjects, preferably expected to have immune reaction and antibodies against the peptides disclosed in the invention.

Screening of peptides. The invention is further directed to screening of the preferred peptide epitopes and analogous peptides and conjugates thereof against human immune reactions for development of the optimal vaccines and antibody development products.

The invention is further directed to further screening of, and binding analysis of peptides, which are recognized by patients immune system preferably by natural antibodies of a patient. The invention is directed to screening methods to reveal further peptides derived from carbohydrate binding proteins (adhesions/lectins) of human pathogens, especially carbohydrate binding sites of pathogens comprising large carbohydrate binding sites involving binding to multiple monosaccharide units, more preferably including binding sites for two sialic acid structures.

Preferred types of influenza viruses. The influenza viruses are preferably viruses involving risk for human infection, including human influenza viruses, and/or potentially human infecting pandemic influenza viruses such as avian influenza viruses. More specifically the preferred virus is influenza A, influenza B and influenza C viruses, even more preferably influenza A or B, and most preferably influenza A. Preferably influenza A is a strain infecting or potentially infecting humans such as strains containing hemagglutinin type H1, H2, H3, H4, or H5.

Preferred Peptides or Groups of Peptides for Influenza Viruses

The invention is directed to specific peptide epitopes and variants thereof for treatment of influenza (including prophylactic or preventive treatments). The invention is specifically directed to specific peptide epitopes and groups thereof for treatment of specific subtypes of influenza such as influenzas involving hemagglutinin types H1, H2, H3, H4, or H5, more preferably H1, H2, H3, or H5, even more preferably H1, H3 or H5. Especially human infecting types of hemagglutinins, especially hemagglutinins H1, H3, and H5 viruses are preferred and even more preferably H1 and H3 are preferred. In an especially preferred embodiment the peptide is conformational peptide 2 and 3, even more preferably peptide 3, from the preferred hemagglutinin types including H1, H3 and H5, H1 and H3 and most preferably H3.

Several Peptides Against the Same Hemagglutinin or Homologous Hemagglutinins

It is realized that part of the sequences comprise relatively fast mutating semiconserved residues. Production of peptides with multiple variants for longer about 20 meric peptides is chemically feasible by standard technologies, see for example influenza patent applications of Variation biotechnology and related background publications. The shorter peptide epitopes according to the present invention are even more effective for synthesis and includes less variants. In a preferred embodiment the peptide composition for binding and selection methods or according to the invention includes variants of the peptides currently present in influenza virus. The preferred and most relevant variants includes 1-5 variants for peptides 1-3, more preferably 1-3 variants or 2 or 3 variants. The amount of variants needed depend on the current status of evolution of the specific peptide, when the peptide is changing from one major variant to another there is multiple variants present typically at least one major old variant e.g. WVR variant of H3 and more recent RVR variants of peptide 3 were present simultaneously. It is further realized that the peptide 2 comprises especially many semiconserved residues and invention is directed to including more variants typically two to five, more preferably 2-4 variants or most preferably at least 2 or 3 variants of it for effective vaccine. Less important residues at N- or C-terminus may be more varying such as N-terminal residue of peptide 3.

In case a linear peptide or a conformational peptide would be considered, preferably by analysis from databases, as autoimmunity causing non-autoimmunogenice variants thereof are selected and/or peptide(s) from another region(s) (peptide 1 or peptide 2 or peptide 3) are included in the vaccine.

The invention is directed to preferred peptide compositions for binding analysis and/or peptide selection, and especially immunization and/vaccination, when the composition comprises at least 2, preferably 2-5, more preferably 2-4, different peptide sequences, preferably conformational sequences according to the invention, which are variants of the same peptide (selected from the group peptide 1, peptide 2 and peptide 3, more preferably peptide 2 and 3). The preferred vaccine composition preferably further comprises a second type of immunogenic peptide, and optionally current variant(s) thereof, from influenza selected from the group:
i) a peptide from different region of hemagglutinin, selected from the group peptide 1, peptide 2 and peptide 3,
ii) a peptide from the same region of hemagglutinin but from different hemagglutinin type (preferably from hemagglutinins H1-H5, more preferably from the preferred hemagglutinins according to the invention and
iii) another known antigenic peptide from
   a. another site of hemagglutinin protein such as the known peptide vaccine epitopes conserved at cleavage site of precursor HA0 from hemagglutinin or other longer hemagglutinin peptides
   b. another protein of influenza virus, preferably a conserved
      i. peptide epitopes of M2 protein
      ii. peptide epitopes of NP protein of influenza

In yet another preferred embodiment the vaccine composition comprises at least two variants of two peptides according to i), preferably peptide 2 and peptide 3 and in yet another preferred embodiment at least additional peptide according to ii) and more preferably at least two peptides according to ii) and most preferably at least one, more preferably at least two variants there of.

It is further realized that relatively good influenza restricting or taming though not effectively blocking responses have been obtained by M2 peptides of influenza, or by HA₀ or NP protein based epitopes and the vaccines and known combinations therefore it would be beneficial to combine with current peptides according to the invention.

In a preferred embodiment the preferred compositions for the methods according to the invention comprises peptide 2 and 3 of two (preferably H1 and H3) or three hemagglutinins (preferably H1, H3 and H5). In specifically preferred embodiment preferably H1 and H3 hemagglutinin and at least one variant of one peptide, more preferably at least one variant of two peptides, and in another preferred embodiment at least one variant of three or all four peptides, and it is especially preferred to include variants of peptide 2, even 3 or more variants, and optionally a least one variant of one peptide 3 preferably H3 type of peptide 3 for vaccination or analysis of current influenza

The invention is further directed to combinations of current peptides with complete hemagglutinin protein or another influenza virus protein or domain there of comprising e.g. about 50-100 amino acid residues, known as potential influenza vaccines and or one influenza viruses or analogous viral particles comprising surface protein(s) of influenza.

The preferred HA0 from hemagglutinin peptides includes e.g. ones developed by Merck and Biondvax and known in background of their publications. Other preferred hemagglutinin peptides from includes e.g. ones developed by Variation biotechnology e.g including peptide 1 and peptide 4 described in WO06128294 (Jul. 12, 2006) and Biondvax including peptide HA91 (e.g. WO07066334, Jun. 14, 2007) directed to longer peptides epitopes which are not conformational and conjugated according to the present invention.

The preferred M2 protein or peptide epitopes are developed by the companies including Merck US (peptides), Acambis (with Flanders Univ.), AlphaVax (with NIH, pandemic), VaxInnate (with Yale Univ.), Dynavax (with support from NIH), Cytos Biotech, CH), GenVec (with NIAID), or Molecular Express, Ligocyte or Globe immune or Biondvax (Israel, Ruth Amon and colleagues) and known from the background of their publications. M2 also referred as M2e is common (conserved) antigen and ion channel on influenza, it is not accessible on viral surface but targeted on infected cells (assembly of virus) and it does not cure effectively but relieve disease (Science 2006, Kaiser).

The preferred NP protein (nucleoprotein of influenza) or peptide epitope are developed e.g. by the companies Biondvax, AlphaVax, GenVec and known from the background of their publications)

Preferred Conserved Amino Acid Epitopes, Antigen Peptides, for Vaccine or Antibody Development

The present invention is preferably directed to following peptide epitopes, and any linear tripeptides or tetrapeptides derivable thereof or combinations thereof for vaccine and antibody development, preferably directed for the treatment of human influenza. The invention is further directed to elongated versions of the peptides containing 1-3 amino acid residues at N- and/or C-terminus of the peptide. The numbering of the peptides is based on the X31-hemagglutinin if not otherwise indicated. This indicated corresponding position of the peptides in three dimensional structure of the hemagglutinin and same position with regard to conserved cysteine bridge for Peptide 1 and Peptide 2 and presence in the loop structure as described for Peptide 3.

The invention is specifically directed to sequencing and analysing corresponding peptides from new influenza strains, because the viruses have tendency to mutate to avoid human immune system. The invention further revealed that it is possible to use several peptides according to the invention. Persons resistant to influenza virus had antibodies against 2 or 3 peptides. The invention is directed to vaccines against single type of influenza H1, H2, H3, H4 or H5.

The invention is further directed to peptide compositions comprising at least one peptide, more preferably at least two and most preferably at least three peptide, against at least two, more preferably at least three, different hemagglutinin subtypes, preferably against H1, H3, and/or H5. In a specific embodiment the invention is directed to peptides of H5-hemagglutinins aimed for treatment or prevention of avian influenza.

It is further realized that similar peptides may be derived from other influenza virus hemagglutinins. The invention is specifically directed to defining structurally same peptide positions from influenza B, Influenza C and other hemagglutinin subtypes such as H6, H7, H8, or H9.

It is further realized that the peptides may be used in combination with known and published/patented peptide vaccines against influenza and/or other influenza drug. The invention is specifically directed to the use of the vaccines together with hemagglutinin binding inhibiting molecules according to the invention, preferably divalent sialosides. The invention is further directed to the use of the molecules together with neuraminidase inhibitor drugs against influenza such as Tamiflu of Roche or Zanamivir of GSK or Peramivir of Biocryst or second generation neuraminidase inhibitors such as divalent ones developed by Sankyo and Biota

The peptides are preferably aimed for use as conjugates as polyvalent and/or immunomodulator/adjuvant conjugates. The preferred epitopes do not comprise in a preferred embodiment additional, especially long amino acid sequences. The length of the short conformational epitopes is preferably less than 13 amino acid, and preferred shorter epitopes, as described for the short epitopes. There are preferably less than 7 amino acid, more preferably less than 5, more preferably less than 3 and most preferably less 1 or 0 additional amino acid residues, directly continuing from the original hemagglutinin sequence.

It is realized one or several of the amino acid residue can be replaced by mimicking residue having similar conformation. The invention is further directed to methods for optimization of the peptides so that part of the sequence, which is preferably analyzed by molecular modeling and/or binding method according to the invention, especially N- and/or C-terminal amino acid residue(s)/additional residues at N- or C-terminus, be changeable to similar residues supporting the conformation of the peptide. The invention is further directed to the optimization of chemical epitopes of the linear and or conformational peptides by standard peptide optimization methods, which in a preferred embodiment includes introduction of structures resistant to proteases and or peptidases present in the patient.

The invention revealed that it is possible to modify the cyclic peptide 3, from both N- and C-terminal ends, the modification produces effective 3D structures to be used according to the invention. In a preferred embodiment
a) the N-terminus of the peptide is conjugated to a spacer or carrier as described here for covalent conjugates, as an example by a C2-C10 alkyl spacer to biotin to be conjugatable to an avidin based matrix (avidin is in a preferred embodiment streptavidin or like and
b) the C-terminus of the peptide is conjugated to an additional amino acid or amino acids or spacer residue to increase the stability of the peptide and/or its accessibility. In a preferred embodiment a small neutral residue, such as glycine amide, is linked to the C-terminus in the cyclic peptide.

The invention is further directed to method for production of the cyclic peptide when at least one elongating residue, preferably a neutral amino acid residue such as glycine amide, is conjugated to the C-terminus and after that step the cyclic peptide is conjugated from N-terminus. to spacer and/or directly to oligo/polyvalent carrier.

Many peptide vaccines have been described against influenza virus. These contain various peptides of the virus usually conjugated to carriers, or other immunogenic peptides and/or adjuvants and further including adjuvant molecules to increase antigenicity.

Person skilled in the art can determine the corresponding amino acid position from other influenza hemagglutinins in relation to most conserved amino acid residues and/or position of disulfide bridges and design similar peptides containing 1-3 different, especially preferred peptide 3, more preferably 1-2 different amino acid residues, most preferably only one different amino acid residue. Design of analogs and elongated variants of the peptides involves analysis of the surface presentation of the peptides, so that these would be accessible for analytic/diagnostic and/or therapeutic recognition by specific binding agents, such as antibodies, peptides (such as phage display peptides), combinatorial chemistry libraries and/or aptamers.

Preferred Hemagglutinin Peptides

Region of Amino Acid at Positions of about 210- to 230 of Hemagglutinin

Similarity is observed between influenza A viruses for example as partial, very short peptide epitope sequence KVR and isoforms in hemagglutinin type H1 sequences and similar positively charged RVR in current strains H3 after about year 2000, WVR in older H3 and KVN in H5. The region is favoured because presence on the surface of the virus available for immune recognition and because antibodies binding to the region would interfere with carbohydrate binding of the virus. The peptides form a conserved loop type epitope which can be further used for production of cyclic peptides. The invention is especially directed to conformational epitopes represented by the cyclic peptide structure.

It is realized that it is useful and preferred to represent the peptide 3 epitopes in a assay and/or binding method as a conjugated form. The background describes passive absorption of peptides but the present invention reveals very effective and robust assay, when the peptides are specifically conjugated covalently and/or by strong non-covalent linkage. The invention is further directed to specifically conjugated or covalently conjugated conformational epitopes represented for the immune system. In a preferred embodiment the invention is directed to conjugated structure, wherein the peptide is conjugated from the N-terminal or C-terminal end of the peptide sequence. In another preferred embodiment the peptide is conjugated only from N-terminal end, the invention revealed that such peptides can be effectively recognized by antibodies. In yet another preferred embodiment the peptide is conjugated from both N-terminal and C-terminal and to solid phase or soluble carrier.

In a preferred embodiment the cyclic peptide is separated from the carrier or solid phase by a linking atom group and/or linking atom group and a spacer.

Preferred KVR-Region Peptides of H1 Similar Peptides

The conserved amino acid (from amino terminus to C-terminus) Lys222-Va1223-Arg224 KVR homologous to WVR-region of X31 hemagglutinin forms an excellent target for recognition of influenza virus. This relatively conserved sequence is present e.g. in the sequence RPKVRDQ of A/South Carolina/1/1918 (H1N1), also known as “Spanish Flu”-hemagglutinin. The peptide was modeled as an exposed sequence on the surface of the virus. The peptide sequence is preserved in hundreds human influenza A viruses. The region comprise a tripeptide Lys222-Va1223-Arg224 (KVR), which is a preferred peptide epitope according to the invention and present in longer peptide epitopes. Preferred peptide epitopes includes heptapeptide RPKVRDQ and further includes pentapeptides: RPKVR, PKVRD, KVRDQ and hexapaptides RPKVRD and PKVRDQ. The proline is preferred as an amino acid affecting the conformation of the peptide, the D-residues is preferred as a semi-conserved amino acid residue, it may be replaced by similar type amino acid residue

Conserved Peptide 3 Region of Hemagglutinin 2, H2

The invention revealed that human hemagglutinin 2 also contains conserved Peptide 3 region the examples of the sequences includes RPEVNGQ and RPKVNGL, the epitope comprises additional amino acid residues K and E—especially at N-terminal side, with consensus sequence RPXVNG or

PXVNG, RPXVN, RPXV, PXVN, XVNG, RPX, PXV, XVN wherein X is any amino acid preferably E or K

Preferred WVR-Region Peptides of H3 Similar Peptides

The conserved amino acid (from amino terminus to C-terminus) Trp222-Va1223-Arg224 WVR of region B of X31 hemagglutinin forms another excellent target for recognition of influenza virus. The peptide was modeled as an exposed sequence on the surface of the virus. The peptide sequence is preserved in more than hundred human influenza A viruses. The region comprise a tripeptide Lys222-Va1223-Arg224 (WVR), which is a preferred peptide epitope according to the invention and present in longer peptide epitopes. Preferred peptide epitopes includes heptapeptide RPWVRGL and further includes pentapeptides: elongated variants pentapeptides, RPWVR, PWVRG, WVRGL and hexapaptides RPWVRG and PWVRGL. The proline is preferred as an amino acid affecting the conformation of the peptide, the L-residues is preferred as a semi-conserved amino acid residue, it may be replaced by similar hydrophobic amino acid residue. The preferred variants include ones where W is replaced by R-residue.

Preferred KVN-Region Peptides of H5 Similar Peptides

The conserved amino acids Lys222-Va1223-Asn224 (KVN, from amino terminus to C-terminus) observable for example from H5-hemagglutinins A/Vietnam/1203/2004 (H5N1) or A/duck/Malaysia/F119-3/97 (H5N3), corresponding to conserved region B of X31 hemagglutinin forms a further target for recognition of influenza virus. The peptide was modeled as an exposed sequence on the surface of the virus. The peptide sequence is preserved in more than hundred human influenza A viruses.

Preferred peptide epitopes further includes elongated variants peptides being the heptapeptide RPKVNGQ, hexapeptides RPKVNG, and PKVNGQ, pentapeptides RPKVN, PKVNG, KVNGQ, RPKVNG, and PKVNGQ. The penta- to hepta peptides all includes the preferred tripeptide structure KVN. The invention is further directed to tetrapeptides RPKV, PKVN, including the preferred subepitope KV and KVNG and VNGQ including preferred subepitope VN. The proline is preferred as an amino acid affecting the conformation of the peptide, it may be replaced by similar type amino acid residue.

The invention is specifically directed to consensus of Peptide 3 region

RPX₁VX₂X₃

X₁ is K, E, R or W

X₂ is N, or R

X3 is noting, D or G.

Cyclic Peptides of the Region about 210-230

The invention is further directed cyclic peptides including the preferred peptide epitopes above. Most preferably a natural type heptapeptides RPKVRDQ, RPWVRGL, RPKVNGQ linked to a cyclic peptide by residues X and Y:

X—H7-Y,

wherein H7 is the heptapeptide and

X is group forming cyclic structure with group Y,

In a preferred embodiment X and Y are Cys-residues forming disulfide bridge With each other.

The groups X and Y include preferably pair of specifically reactive groups such as amino-oxy (—R—O'NH2) and reactive carbonyl such as aldehyde or ketone; azide (—R—N═NH2) and reactive carbonyl such as aldehyde or ketone

Region of Amino Acid at Positions of about 85- to about 100/98-106

Similarity is observed between influenza A viruses within a region corresponding to the amino acids located before cysteine 97 in the structure of H3 hemagglutinin X31. The region is favoured because presence on the surface of the virus available for immune recognition and because antibodies binding to the region would interfere with carbohydrate binding of the virus. The region is mainly semiconserved, there is similar variants of the sequences, which are relatively well conserved within each hemagglutinin type.

Analysis of Consensus Sequences by a Group of H1-H5 Viruses Form Animals and Human

Peptide 1

The peptide 1 sequences were revealed to be present as four major groups A, B, C and D

The consensus sequence for peptide Peptide 1 group A is

K A₁ N₂ P A₃ N₄ D₅ L C

wherein A₁ is A, D, E, I, or T; N₂ is N, S, or T; A₃ is A or V, I, D, R or K; N₄ is N, Y or D; D₅ is D, G or S. Additionally in a variant C may replace sub-carboxyterminal L and amino-terminal K may be replaced by the similar positive charged R. The group A can be further divided to two groups A1 and A1

subgroup A1, wherein A₃ is positively charged residue, preferably R or K, and A₁ is negatively charged residue, preferably E

subgroup A2, wherein A₃ is hydrophobic alkyl-side chain residue, preferably A, V or I, and A₁ is negatively charged residue, preferably E, or D, or hydrophobic residue A; and/or N₂ is optionally S or T

The consensus sequence for peptide Peptide 1 group B is

T S₁ N₂ S₃ E₄ N₅ G T₅ C

wherein S₁ is S, R, or P; N₂ is N, or T; S₃ is S or P; E₄ is charged residue E, K or D; N₅ is N or T; T₅ is T, A or I.

Preferred subgroups of B includes B1 with S₃ is S and B2 wherein S₃ is P having clear conformational differences due to structure of P. In a preferred embodiment N₅ is N, which is common residue in peptides B.

The consensus sequence for peptide Peptide 1 group C is

R P N₁ A₂-I₃ D T C

wherein N₁ is N, or T; A₂ is A, or T; 1₃ is hydrophobic aliphatic residue, preferably branched residue, more preferably V or I. This group form a specific group of hemagglutinins with preferred PrePeptide 1 comprising D V F I or very homologous residues.

The consensus sequence for peptide Peptide 1 group D is

R S N₁ A-F₂ S N₃ C

wherein N₁ is N, K, or T; F₂ is an aromatic side chain amino acid, preferably F, or Y; N₃ is polar residue, preferably N, D, S or T. This group form a specific group of hemagglutinins with preferred PrePeptide 1 comprising D L F or very homologous residues.

It is realized that additional few amino acid residues may be included to amino or carboxy-terminal to improve conformation of the peptide. The elongated peptides may be more useful for database searches. The preferred carboxyterminal additional amino acid residue includes 1-6, more preferably 2-4 and most preferably 3 or 4 amino acid residue consecutive to the peptide 1.

Total Consensus of Peptide 1

The total consensus sequence for peptide Peptide 1 is

R₁ S₂ N₃ A₄ E₅ N₆ G₇ N₈ C

wherein

R₁ is a polar positively charged or non-charged residue preferably from group R, K, or T;

S₂ is polar residue S, or T; N or D or R: or conformational residue P

N₃ is polar residue S, or T; N or K.

A₄ is polar residue S, or T; or aliphatic small chain A or conformational residue P.

E₅ is polar residue with negative charge E or D, positive charge R or K; or hydrophobic A, V or I or deleted.

N₆ polar residue N, or D; aromatic F or Y; or hydrophobic residue I or V

G₇ is polar residue G, D or S.

N₈ is polar residue S or T, N, or D; or hydrophobic residue A or L.

It is notable that S or T in position 2, 3, 4 and 8 are very similar with polar hydroxyl side chain, T (and thus putatively also S) can be present in position 1 and S in 7; polar positively charged R and K with similar sizes were both observed position 1, 5 and one of these in 2 and 3; negatively charged similar D and E both in position 5 and D in 2, 6, 7, 8 and amide of D derivative N(positions 2, 3, 6 and 8); at least hydrophobic aliphatic A, V, L, I are present in 4, 5, 6, and 8; and aromatic similar Y and F in position 6.

Referring positive +, negative −, polar O, P-proline, C-hydrophobic alkyl, B-aromatic) (+O), (+−O P), (+O), (O C P), (+−C or del), (−O B C), (−O), (−O C), revealing relatively limited actual variation.

Peptide 3 from Animal and Human H1-H5 Peptide Search

Total Consensus Peptide

R₁ P₂ K₃ V₄ R₅ G₆ Q₇

wherein

R₁ is a polar positively charged group R, K, or non polar small G; or rarely S or I

P₂ is polar residue S, or conformational residue P or hydrophobic L

K₃ is polar charged residue R or K, E or aromatic non-polar residue W.

V₄ is aliphatic hydrophobic amino acid residue A, V, or I.

R₅ is positively charges R or K; or polar N or S.

G₆ similar polar/negative residue N, or D or E; or small polar G,

Q₇ is polar residue Q, or aliphatic hydrophobic amino acid residue V, L or I.

Referring positive +, negative −, polar O, P-proline, C-hydrophobic alkyl, B-aromatic) (+O C), (O P C), (+−B), C, (+O), (−O), (O C), revealing relatively limited actual variation.

The peptide 3 sequences were revealed to be present as three major groups A, B, and C.

The Consensus Sequence for Peptide Peptide 3 Group A is

R₁ P K₂ V R G₆ Q₇

wherein

R₁ is a polar positively charged group R, K, or non polar small G;

K₂ is polar charged residue R or K, E or aromatic non-polar residue W.

G₆ similar polar/negative residue N, or D.

Q₇ is polar residue Q, or aliphatic hydrophobic amino acid residue V, L or I.

The group B is homogeneous group of hemagglutinins with characteristic PrePeptide 3 and especially PostPeptide 3 structures.

The Consensus Sequence for Peptide Peptide 3 Group B is

R P₁ K₂ V N₃ G Q₄

Wherein

P₁ is polar residue S, or conformational residue P or hydrophobic L

K₂ is polar charged residue K or E

N₃ is positively charges R or K; or polar N or S.

Q₄ is polar residue Q, or aliphatic hydrophobic amino acid residue L.

The group B is homogeneous group of hemagglutinins with characteristic PrePeptide 3 and especially PostPeptide 3 structures.

The Consensus Sequence for Peptide Peptide 3 Group C is

R P K V₁ R₂ G₃ Q₄

wherein

V₁ is aliphatic hydrophobic amino acid residue A, V, or I.

R₂ is positively charges R or K.

G₃ similar polar/negative residue N, or D or E; or small polar G,

Q₄ is polar residue Q, or aliphatic hydrophobic amino acid residue L.

The group B is homogeneous group of hemagglutinins with characteristic PrePeptide 3 and especially PostPeptide 3 structures.

Analysis of H3 HA-Consensus Sequences from a Group of Influenza Viruses

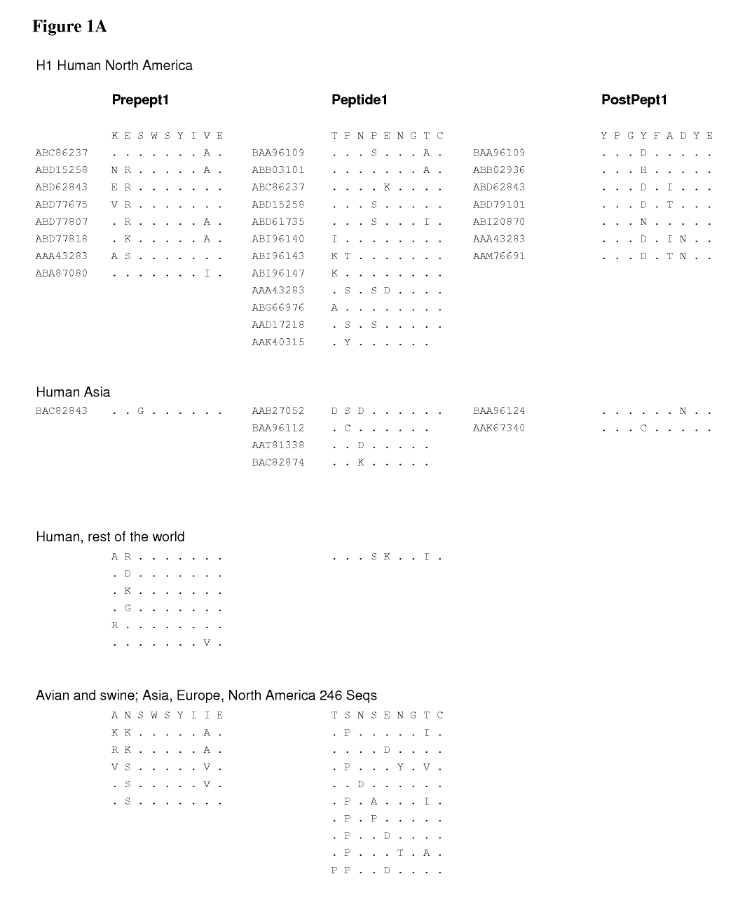

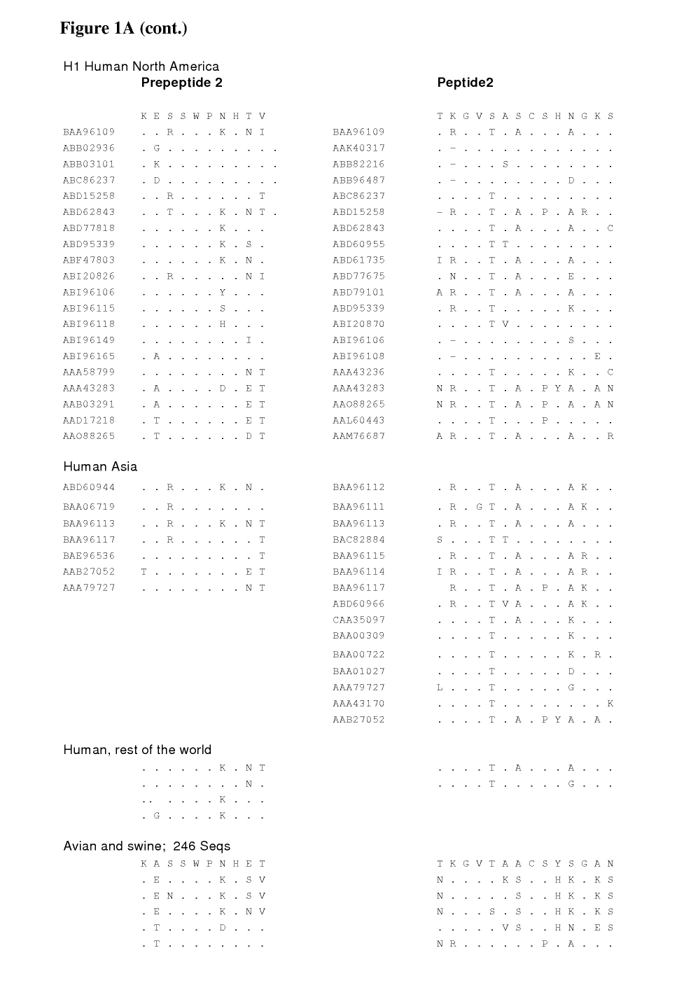

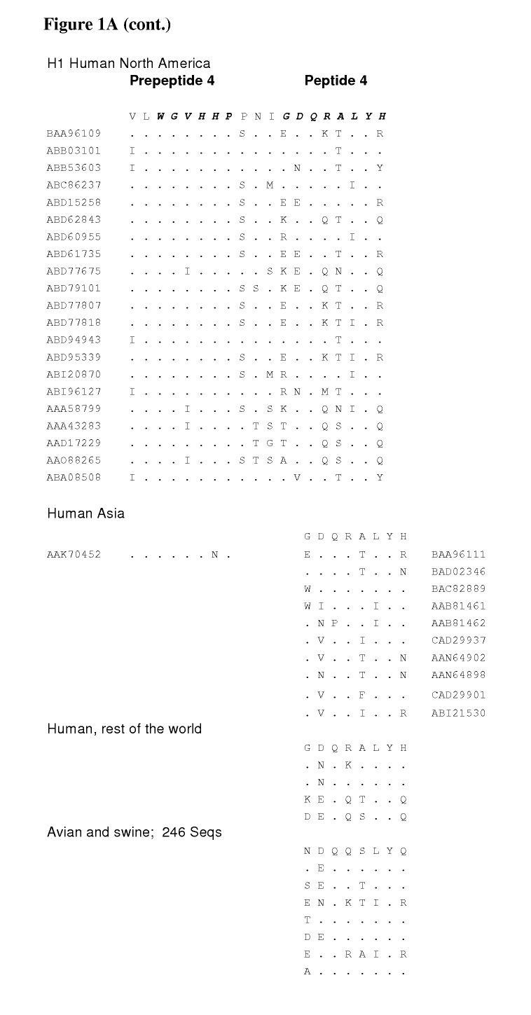

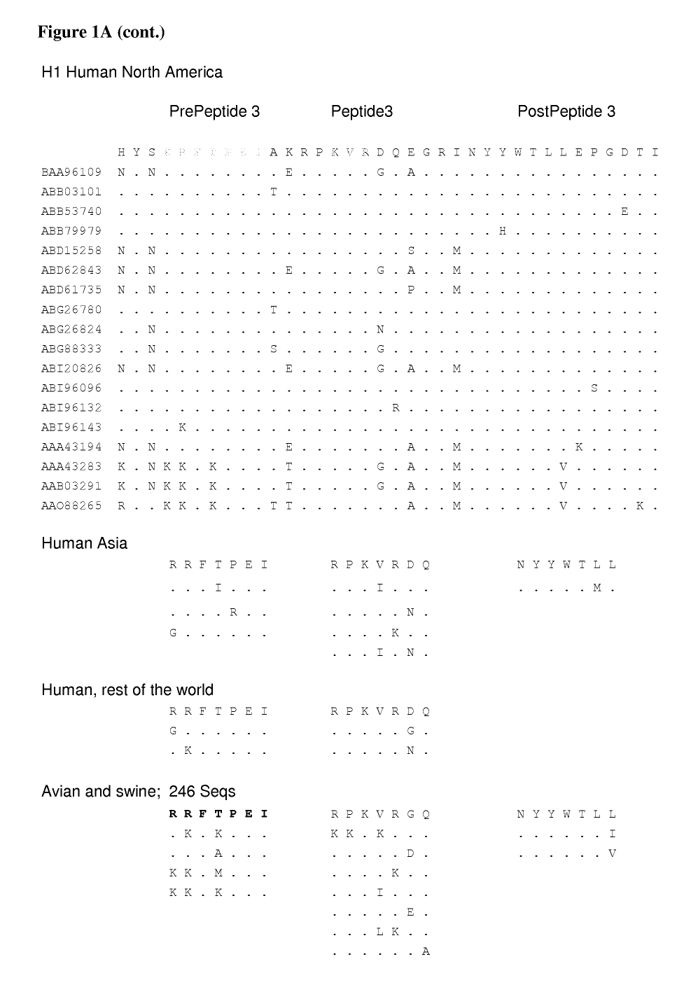

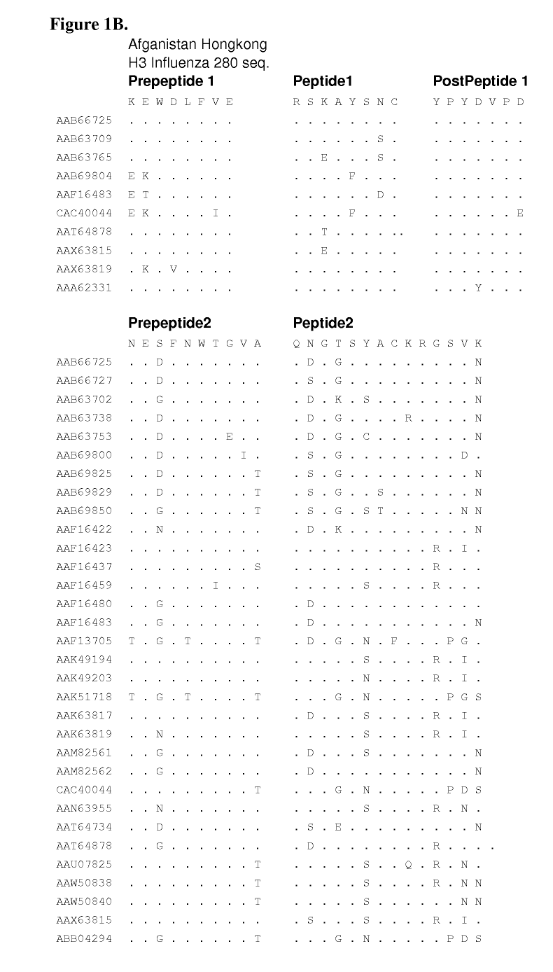

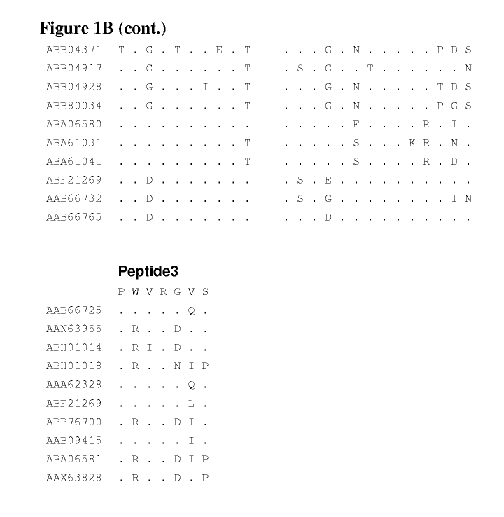

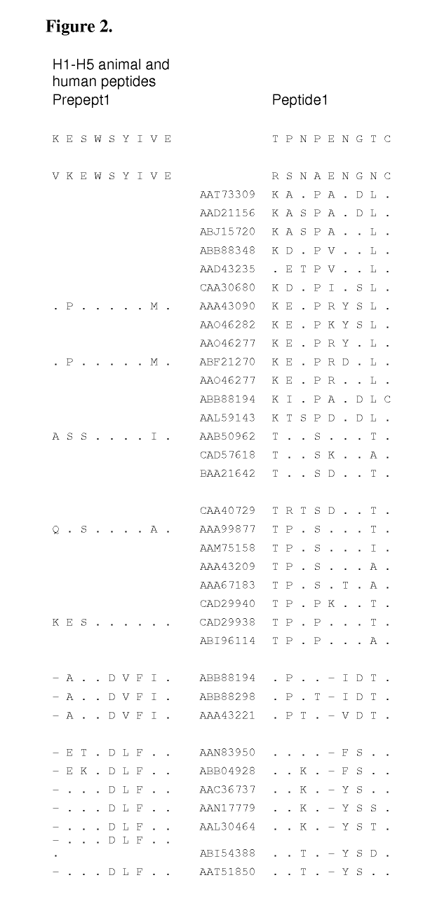

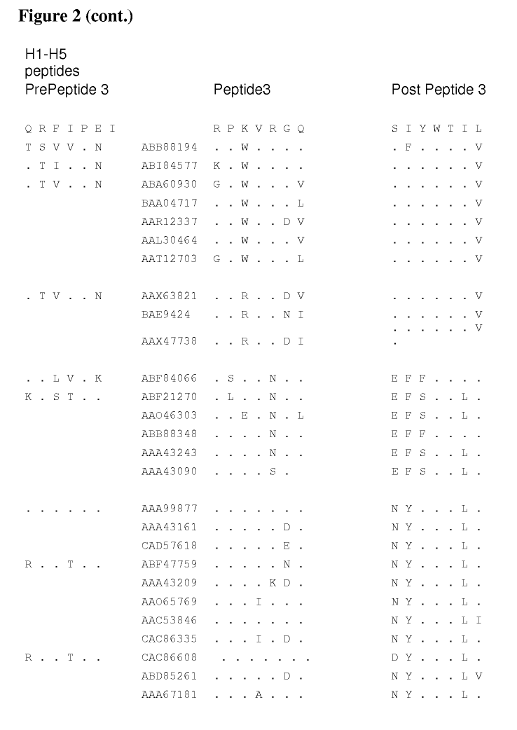

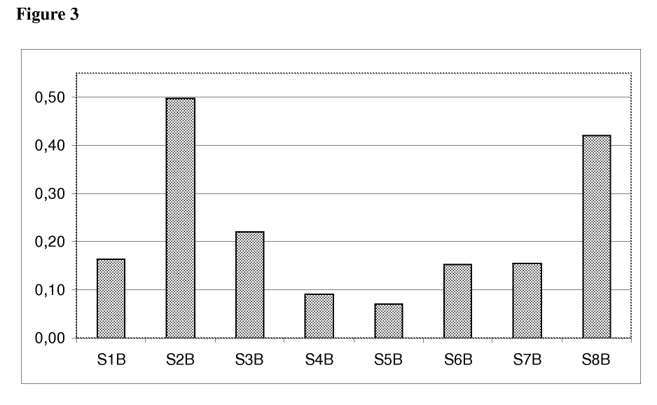

280 H3 sequences was collected and aligned from databank, FIG. 1b. The sample sequences were from Hong Kong and Afghanistan, selected as remote places and remote from Finland which was analyzed separately and part of the sequences were added to the consensus. The aligned sequences were compared in order to reveal consensus sequences and collect individual sequence variants.

The invention is especially directed to collecting and grouping of sequence variants in order to classify viruses and reveal groups of viruses with specific antigenic and other functional such as sialylated natural glycan binding properties as studied in the previous applications of the inventors.

Total Consensus of Peptide 1

The peptides appeared to be homologous, with minor changes

The total consensus sequence for peptide Peptide 1 is

R S K₁ A Y₂ S N₃ C

wherein

K₁ is a polar charged or non-charged residue preferably from group E, K, or T;

Y₂ is aromatic residue Y or F or D(from analysis of Finnish sequences).

N₃ is polar residue S; N or D.

Preferred subgroups of Peptide 1 includes 4 groups A, B C and D

The group A consist of sequences

R S K A Y S N₃ C

wherein the polar residue N3 varies as above

This is a characteristic sequence in many recent viruses

The group B consist of sequences

R S K A F S N C

which is a characteristic sequence in many viruses.

The group C consist of sequences

R S K₁ A Y S N₃ C

wherein the polar residue N3 varies as above and

K₁ is E or T

The group D consist of unusual sequences

R S K₁ A D S N₃ C

wherein the polar residue N₃ varies as above and

K₁ is as above, or these are more preferably N and K, respectively

Peptide 2 of H3 Viruses

Analysis of Finnish sequences gave consensus core peptide sequences SNACKR, SYAKR and SSACKR These the core peptides were compared to ones obtained from the analysis of Hong Kong/Afghanistan viruses The core epitope was elongated by four amino acid residues to include conserved and binding functional residues and by one residue from carboxy-terminus, further residues in the close region are in the Figures

Q N₁ G T₂ S Y₃ A₄ C K₅ R₆ G₇

wherein

N₁ is a polar negatively charged or non-charged residue preferably from group D, N and S,

T₂ is polar neutral or charged residue T, G; D, E or K.

Y₃ is polar residue S,N, or C; or aromatic Y or F

A₄ is aliphatic small chain A or similar polar residue S, or T

K₅ is polar residue with positive charge K or R;

R₆ polar residue with positive charge R or K; preferably R

G₇ is polar residue G, or positively charged, preferably R.

Preferred variant groups includes peptides with different

Y₃, in four groups

Group A according to formula above, wherein Y₃ is N. This is present in old and some new viruses.

Group B according to formula above, wherein Y₃ is Y or F. This is characteristic with residue Yin part of new/90's influenza viruses as in analysis of Finnish viruses.

Group C according to formula above, wherein Y₃ is S. This is characteristic in especially for a group of new influenza viruses observed especially after year 2000 as shown in analysis of Finnish viruses

Peptide 3 of H3 Influenza Viruses

Analysis of Finnish influenza viruses revealed RPWVRGL, RPWVRGV, RPWVRGI, RPWVRGQ, RPRVRD(V/I/X). The Afghanistan/Hong Kong viruses were analyzed including one additional residue at carboxy terminus of the core sequence, as preferred additional residue.

Consensus sequence of H3 influenza peptides

R₁ P W₂ V₃ R G₄ V₅ S₆

wherein

R₁ is a polar positively charged group preferably R, or other G, S or I;

W₂ is large aromatic hydrophobic W or positively charged group. preferably R

V₃ is alkyl hydrophobic residue, preferably V or I.

G₄ is polar residue G, N or D

V₅ is non-charged Q or hydrophobic V, L or I.

S₆ is polar S or conformational P.

Preferred structure groups include common according to the consensus Formula:

Group A wherein R₁ is R and

More rare

group B wherein R₁ is not R and is preferably G, S or I.

The preferred structure

Group C includes Structures according to the consensus Formula above wherein

W₂ is W.

and

Group D includes peptides according to the consensus Formula, wherein

W₂ is not W, preferably being positively charged residue, more preferably R, and also preferably

G₄ is not G, and preferably G₄ is D or N.

Antigenic Compound

An “antigenic compound” as used herein means a compound, for example a peptide, or a composition of multiple, two or three or more peptides, or peptide like compounds, which can elicit an antigenic reaction in an animal. It is not necessary for an antigenic compound to elicit or raise an immunogenic reaction; it may do so or not. An antigenic compound may be used for the purposes of raising immunogenic response or for screening assays. An antigenic compound comprises an epitope or epitopes which may be or are suitable for eliciting an immunogenic response. Favorably, an antigenic compound, for example, a peptide or peptides conjugated to together, via a peptide sequence or by other means, e.g. covalently, binds an antibody substance and can elicit an immunogenic response in a mammalian subject, e.g. in humans. An antigenic compound can be used in in vitro assays, for example in binding assays when screening antibody substances which bind an antigenic compound or compounds.

Preferably an antigenic compound comprises a peptide and said peptide corresponding to influenza virus A hemagglutinin. “Corresponds” as used herein means that the amino acids of an antigenic compound are similar or homologous to influenza virus A hemagglutinin amino acids. Skilled artisan understands when peptide of the present invention corresponds influenza virus A hemagglutinin and when the peptide or the antigenic compound is something else than HA or influenza virus A.

In more preferred embodiment the peptide K₁V₂R₃ according to claims, wherein K₁ is an optional residue of an amino acid selected from the group of K, E, M and conservative substitutes thereof; V₂ stands for a residue of an amino acid selected from the group of V, I, L, F, A and conservative substitutes thereof; and R₃ is a residue of an amino acid selected from the group of R, K and N and conservative substitutes thereof.

In more preferred embodiment the peptide W₁V₂R₃ according to claim 1, wherein Wᵢ is an optional residue of an amino acid selected from the group of W, R, L, K and conservative substitutes thereof; V₂ stands for a residue of an amino acid selected from the group of V, I, A, E, G and conservative substitutes thereof; and R₃ is a residue of an amino acid selected from the group of R and conservative substitutes thereof.

In more preferred embodiment the peptide K₁V₂N₃ according to claim 1, wherein K₁ is an optional residue of an amino acid selected from the group of K, E, R, Q, M and conservative substitutes thereof; V₂ stands for a residue of an amino acid selected from the group of V, I, L, F, A and conservative substitutes thereof; and N₃ is a residue of an amino acid selected from the group of N, R, K, D and conservative substitutes thereof.

An antigenic compound comprises preferably 5 to 13 amino acids. The antigenic compound can be shorter, e.g. 3 or 4 amino acids, or it can be longer, 6, 7, 8, 9, 10, 11, or 12 amino acids. The prior art teaches long antigenic peptides derived from influenza virus A but in the present invention inventors have discovered that short amino acid sequences are better to e.g. screen natural antibodies and elicit an immunogenic response.

The preferred influenza virus A hemagglutinin subtypes according to invention are hemagglutinin (HA) subtypes H1, H3 and H5. Even more preferred subtypes are H1N1, H3N2 and H5N1.

Preferably an antigenic compound comprises at least two peptides as defined in claim 1. In some applications it is beneficial to include two peptides, which together enhance the binding efficiency of an antibody substance and inhibition of influenza virus binding to epithelial cells or target cells influenza virus infects. An antigenic compound comprising at last two peptides is even more preferred antigenic compound.

In more preferred embodiment the antigenic compound comprises at least three peptides as defined in claim 1. In some applications it is beneficial to include three peptides, which together enhance the binding efficiency of an antibody substance and inhibition of influenza virus binding to epithelial cells or target cells influenza virus infects. Antibody substances binding to or recognizing three peptides of the present invention are potent inhibitors of influenza virus. An antigenic compound comprising at last three peptides is preferred antigenic compound of the present invention.

The present invention embraces also a method for producing a vaccine against influenza virus. Preferred steps comprise preparing an antigenic compound comprising at least one peptide according to claim 1; administering said compound to an animal; and monitoring the animal in order to detect immune response against the antigenic compound.

In more preferred embodiment an antigenic compound comprises at least two peptides according to claim 1.

Preferably, an antigenic compound used for a vaccine comprises a carrier, other immunogenic peptides, or an adjuvant. Even more preferably, the peptide is covalently linked to the surface of a carrier protein.

The invention contemplates a vaccine composition comprising an antigenic compound.

Vaccination is preferably performed before anticipated influenza virus infection in a mammalian or human subject. Vaccination can also be done for other animal hosts of influenza virus, e.g. avian or swine species. By this mean eradication or prevention of influenza virus spread in animal populations is prevented or diminished.

Invention also contemplates a method for screening a binding agent against influenza virus HA. Screening method comprises steps of selecting an antigenic compound according to claim 1, assaying binding between antigenic compound and the binding agent; and monitoring the binding of the antigenic compound and binding agent.

The present invention contemplates a method of identifying influenza virus in a biological sample, the method comprising: (a) contacting the biological sample with an antibody substance capable of binding antigenic compound according to claim 1; and (b) detecting the binding between said antibody substance and antigenic compound in the sample, said binding indicating the presence and type of influenza virus in the sample.

The above method is preferred method for detecting influenza virus A HA in a sample.

Binding agent can be an antibody substance as described herein. Binding agent can be a sugar molecule and the binding assay can comprise a modulatory agent, e.g. sugar or oligosaccharide that binds to HA or target cells of HA binding, and effect of modulatory agent is monitored on binding between antigenic compound and binding agent Skilled artisan know several in vitro and in vivo methods to assay screening of binding agents and binding between binding agent and antigenic compound of the present invention. Exemplary assays are represented in U.S. Pat. No. 7,067,284, U.S. Pat. No. 7,063,943 by Cambridge Antibody Tech, WO2006055371, US2006205089 by Univ. Montana, which are incorporated here in their entirety.

Preferably, binding agents for a library, e.g. antibody library or phage display library, and an antigenic compound is exposed to constituents of the library in conditions favorable for interaction between binding agent and antigenic compound. Libraries of the present invention comprise phage display libraries in which antigenic compounds of the present invention are incorporated or antibody libraries, e.g. U.S. Pat. No. 7,067,284, U.S. Pat. No. 7,063,943 by Cambridge Antibody Tech, WO2006055371. In more preferred embodiment antibody substance is a human antibody, preferably IgM and/or IgG. In more preferred embodiment screening is performed in human serum. By this mean natural antibodies from a human subject or subjects can be assayed and/or screened. Further, these antibodies can be sequenced, produced and administered to human patients infected by an influenza virus or before anticipated infection.

The present invention also

The term “amino acid” as used herein means an organic compound containing both a basic amino group and an acidic carboxyl group. Included within this term are natural amino acids (e.g., L-amino acids), modified and unnatural amino acids (e.g. β-alanine), as well as amino acids which are known to occur biologically in free or combined form but usually do not occur in proteins. Included within this term are modified and unusual amino acids, such as those disclosed in, for example, Roberts and Vellaccio, 1983, the teaching of which is hereby incorporated by reference. Genetically coded, “natural” amino acids occurring in proteins include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline, and valine. Natural non-protein amino acids include, but are not limited to arginosuccinic acid, citrulline, cysteine sulfmic acid, 3,4-dihydroxyphenylalanine, homocysteine, homoserine, ornithine, 3 -monoiodo tyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-Cbz-protected amino acid, 2,4-diaminobutyric acid, homoarginine, norleucine, N-methylaminobutyric acid, naphthylalanine, phenylglycine, 9-phenylproline, tert-leucine, 4-aminocyclohexylalanine, N-methyl-norleucine, 3,4-dehydroproline, N,N-dimethyl-aminoglycine, N-methylaminoglycine, 4-aminopiperidine-4-carboxylic acid, 6-amino-caproic acid, trans-4-(aminomethyl)-cyclohexanecarboxylic acid, 2-, 3-, and 4-(amino˜methyl)-benzoic acid, 1-aminocyclopentanecarboxylic acid, 1-aminocyclopropane-carboxylic acid, and 2-benzyl-5-aminopentanoic acid.

Generally, “peptide” stands for a strand of several amino acids bonded together by amide bonds to form a peptide backbone. The term “peptide”, as used herein, includes compounds containing both peptide and non-peptide components, such as pseudopeptide or peptidomimetic residues or other non-amino acid components. Such a compound containing both peptide and non-peptide components may also be referred to as a “peptide analog”.

The terms “conservative substitution” and “conservative substitutes” as used herein denote the replacement of an amino acid residue by another, biologically similar residue with respect to hydrophobicity, hydrophilicity, cationic charge, anionic charge, shape, polarity and the like. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of argmine for lysine, glutamic acid for aspartic acid, or glutamine for asparagine, and the like. Neutral hydrophilic amino acids, which can be substituted for one another, include asparagine, glutamine, serine and threonine. The term “conservative substitution” also includes the use of a substituted or modified amino acid in place of an unsubstituted parent amino acid provided that substituted peptide reacts with hK2. By “substituted” or “modified” the present invention includes those amino acids that have been altered or modified from naturally occurring amino acids.

Administration of the compositions can be systemic or local and may comprise a single site injection of a therapeutically effective amount of the peptide composition of the present invention. Any route known to those of skill in the art for the administration of a therapeutic composition of the invention is contemplated including for example, intravenous, intramuscular, subcutaneous or a catheter for long-term administration. Alternatively, it is contemplated that the therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly.

The peptides of the invention will be used as therapeutic or vaccine compositions either alone or in combination with other therapeutic agents. For such therapeutic uses small molecules are generally preferred because the reduced size renders such peptides more accessible for uptake by the target. It is contemplated that the preferred peptides of the present invention are from about 6, 7, 8, 9, or 10 amino acid residues in length to about 90 or 100 amino acid residues in length. Of course it is contemplated that longer or indeed shorter peptides also may prove useful. Thus, peptides of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 and a 100 amino acids in length will be particularly useful. Such peptides may be present as individual peptides or may coalesce into dimers or multimers for greater efficacy.

The polypeptides of the invention include polypeptide sequences that have at least about 99%, at least about 95%, at least about 90%, at least about 85%, at least about 80%, at least about 75%, at least about 70%, at least about 65%, at least about 60%, at least about 55%, at least about 50%, or at least about 45% identity and/or homology to the preferred polypeptides of the invention, the GDNF precursor-derived neuropeptides or homologs thereof.

An “antibody substance” as used herein refers to any antibody or molecule comprising all or part of an antigen-binding site of an antibody and that retains immunospecific binding of the original antibody. Antibody-like molecules such as lipocalins that do not have CDRs but that behave like antibodies with specific binding affinity for the peptides of the present invention also can be used to practice this invention and are considered part of the invention. Antibody substances of the invention include monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention, fragments of the foregoing, and polypeptide molecules that include antigen binding portions and retain antigen binding properties. As described herein, antibody substances can be derivitized with chemical modifications, glycosylation, and the like and retain antigen binding properties.

Peptide Vaccines and Use Thereof

Peptide Vaccine Compositions

Peptides can be produced using techniques well known in the art. Such techniques include chemical and biochemical synthesis. Examples of techniques for chemical synthesis of peptides are provided in Vincent, in Peptide and Protein Drug Delivery, New York, N.Y., Dekker, 1990. Examples of techniques for biochemical synthesis involving the introduction of a nucleic acid into a cell and expression of nucleic acids are provided in Ausubel, Current Protocols in Molecular Biology, John Wiley, and Sambrook, et in Molecular Cloning, A Laboratory Manual, Cold Spring Harbor Laboratory Press, 1989.

The application discloses a method of inducing an immune response against cyclic peptide 3 or its conjugate or a corresponding peptide of region B of X31 hemagglutinin. This can be accomplished by conjugating the peptide with a carrier molecule prior to administration to a subject.

In the methods disclosed herein, an immunologically effective amount of one or more immunogenic peptides derivatized to a suitable carrier molecule, e.g., a protein is administered to a patient by successive, spaced administrations of a vaccine composed of peptide or peptides conjugated to a carrier molecule, in a manner effective to result in an improvement in the patient's condition.

In an exemplary embodiment, immunogenic peptides are coupled to one of a number of carrier molecules, known to those of skill in the art. A carrier protein must be of sufficient size for the immune system of the subject to which it is administered to recognize its foreign nature and develop antibodies to it.

In some cases the carrier molecule is directly coupled to the immunogenic peptide. In other cases, there is a linker molecule inserted between the carrier molecule and the immunogenic peptide.

In one exemplary embodiment, the coupling reaction requires a free sulfhydryl group on the peptide. In such cases, an N-terminal cysteine residue is added to the peptide when the peptide is synthesized.

In an exemplary embodiment, traditional succinimide chemistry is used to link the peptide to a carrier protein. Methods for preparing such peptide:carrier protein conjugates are generally known to those of skill in the art and reagents for such methods are commercially available (e.g., from Sigma Chemical Co.). Generally about 5-30 peptide molecules are conjugated per molecule of carrier protein.

Exemplary carrier molecules include proteins such as keyhole limpet hemocyanin (KLH), bovine serum albumin (BSA), flagellin, influenza subunit proteins, tetanus toxoid (TT), diphtheria toxoid (DT), cholera toxoid (CT), a variety of bacterial heat shock proteins, glutathione reductase (GST), or natural proteins such as thyroglobulin, and the like. One of skill in the art can readily select an appropriate carrier molecule.

In a preferred embodiment an immunogenic peptide is conjugated to diphtheria toxin (DT).

In some cases, the carrier molecule is a non-protein, such as Ficoll 70 or Ficoll 400 (a synthetic copolymer of sucrose and epichlorohydrin), a polyglucose such as Dextran T 70.

Another preferred category of carrier proteins is represented by virus capsid proteins that have the capability to self-assemble into virus-like particles (VLPs). Examples of VLPs used as peptide carriers are hepatitis B virus surface antigen and core antigen (Pumpens et al., “Evaluation of and frCP virus-like particles for expression of human papillomavirus 16 E7 oncoprotein epitopes”, Intervirology, Vol. 45, pp. 24- 32,2002), hepatitis E virus particles (Niikura et al., “Chimeric recombinant hepatitis E virus-like particles as an oral vaccine vehicle presenting foreign epitopes”, Virology, Vol. 293, pp. 273- 280,2002), polyoma virus (Gedvilaite et al., “Formation of Immunogenic Virus-like particles by inserting epitopes into surface-exposed regions of hamster polyomavirus major capsid protein”, Virology, Vol. 273, pp. 21-35,2000), and bovine papilloma virus (Chackerian et al., “Conjugation of self-antigen to papillomavirus-like particles allows for efficient induction of protective autoantibodies”, J. Clin. Invest., Vol. 108 (3), pp. 415-423,2001). More recently, antigen-presenting artificial VLPs were constructed to mimic the molecular weight and size of real virus particles et al.,“Construction of artificial virus-like particles exposing HIV epitopes and the study of their immunogenic properties”, Vaccine, pp. 386-392,2003).

A peptide vaccine composition may comprise single or multiple copies of the same or different immunogenic peptide, coupled to a selected carrier molecule. In one aspect of this embodiment, the peptide vaccine composition may contain different immunogenic peptides with or without flanking sequences, combined sequentially into a polypeptide and coupled to the same carrier. Alternatively, immunogenic peptides, may be coupled individually as peptides to the same or a different carrier, and the resulting immunogenic peptide-carrier conjugates blended together to form a single composition, or administered individually at the same or different times.

For example, immunogenic peptides may be covalently coupled to the diphtheria toxoid (DT) carrier protein via the cysteinyl side chain by the method of Lee A. C. J., et al., 1980, using approximately 15-20 peptide molecules per molecule of diphtheria toxoid (DT).

In general, derivatized peptide vaccine compositions are administered with a vehicle. The purpose of the vehicle is to emulsify the vaccine preparation. Numerous vehicles are known to those of skill in the art, and any vehicle which functions as an effective emulsifying agent finds utility in the present invention. One preferred vehicle for administration comprises a mixture of mannide monooleate with squalane and/or squalene. Squalene is preferred to squalane for use in the vaccines of the invention, and preferably the ratio of squalene and/or squalane per part by volume of mannide monooleate is from about 4:1 to about 20:1.

To further increase the magnitude of the immune response resulting from administration of the vaccine, an immunological adjuvant is included in the vaccine formulation. Exemplary adjuvants known to those of skill in the art include water/oil emulsions, non-ionic copolymer adjuvants, e.g., CRL 1005 (Optivax; Vaxcel Inc., Norcross, Ga.), aluminum phosphate, aluminum hydroxide, aqueous suspensions of aluminum and magnesium hydroxides, bacterial endotoxins, polynucleotides, polyelectrolytes, lipophilic adjuvants and synthetic muramyl dipeptide (norMDP) analogs. Preferred adjuvants for inclusion in an peptide vaccine composition for administration to a patient are norMDP analogs, such as N-acetyl-nor-muranyl-L-alanyl-D-isoglutamine, N-acetyl-muranyl -(6-0-stearoyl)-L-alanyl-D-isoglutamine, and N-Glycol-muranyl-L.alphaAbu-D-isoglutamine (Ciba-Geigy Ltd.). In most cases, the mass ratio of the adjuvant relative to the peptide conjugate is about 1:2 to 1:20. In a preferred embodiment, the mass ratio of the adjuvant relative to the peptide conjugate is about 1:10. It will be appreciated that the adjuvant component of the peptide vaccine may be varied in order to optimize the immune response to the immunogenic epitopes therein.

Just prior to administration, the immunogenic peptide carrier protein conjugate and the adjuvant are dissolved in a suitable solvent and an emulsifying agent or vehicle, is added.

Suitable pharmaceutically acceptable carriers for use in an immunogenic proteinaceous composition of the invention are well known to those of skill in the art. Such carriers include, for example, phosphate buffered saline, or any physiologically compatible medium, suitable for introducing the vaccine into a subject.

Numerous drug delivery mechanisms known to those of skill in the art may be employed to administer the immunogenic peptides of the invention. Controlled release preparations may be achieved by the use of polymers to complex or absorb the peptides or antibodies. Controlled delivery may accomplished using macromolecules such as, polyesters, polyamino acids, polyvinyl pyrrolidone, ethylenevinylacetate, methylcellulose, carboxymethylcellulose, or protamine sulfate, the concentration of which can alter the rate of release of the peptide vaccine.

In some cases, the peptides may be incorporated into polymeric particles composed of e.g., polyesters, polyamino acids, hydrogels, polylactic acid, or ethylene vinylacetate copolymers. Alternatively, the peptide vaccine is entrapped in microcapsules, liposomes, albumin microspheres, microemulsions, nanoparticles, nanocapsules, or macroemulsions, using methods generally known to those of skill in the art.

Vaccination

The vaccine of the present invention can be administered to patient by different routes such as intravenous, intraperitoneal, subcutaneous, intramuscular, or orally. A preferred route is intramuscular or oral. Suitable dosing regimens are preferably determined taking into account factors well known in the art including age, weight, sex and medical condition of the subject; the route of administration; the desired effect; and the particular conjugate employed (e.g., the peptide, the peptide loading on the carrier, etc.). The vaccine can be used in multi-dose vaccination formats.

It is expected that a dose would consist of the range of to 1.0 mg total protein/peptide conjugate. In an embodiment of the present invention the range is from 0.1 microg to 1.0 mg, more preferably 0.1 mg to 1.0 mg. However, one may prefer to adjust dosage based on the amount of peptide delivered. In either case these ranges are guidelines. More precise dosages should be determined by assessing the immunogenicity of the conjugate produced so that an immunologically effective dose is delivered. An immunologically effective dose is one that stimulates the immune system of the patient to establish a level immunological memory sufficient to provide long term protection against disease caused by infection with influenza virus. The conjugate is preferably formulated with an adjuvant.

The timing of doses depend upon factors well known in the art. After the initial administration one or more booster doses may subsequently be administered to maintain antibody titers. An example of a dosing regime would be a dose on day 1, a second dose at or 2 months, a third dose at either 4,6 or 12 months, and additional booster doses at distant times as needed.

A patient or subject, as used herein, is an animal. Mammals and birds, particularly fowl, are suitable subjects for vaccination. Preferably, the patient is a human. A patient can be of any age at which the patient is able to respond to inoculation with the present vaccine by generating an immune response. The immune response so generated can be completely or partially protective against disease and debilitating symptoms caused by infection with influenza virus.

Evaluation of the Immune Response

In one aspect, the invention provides a means for classifying the immune response to peptide vaccine, e.g., 9 to 15 weeks after administration of the vaccine; by measuring the level of antibodies against the immunogenic peptide of the vaccine.

The invention thus includes a method of monitoring the immune response to the peptide(s) by carrying out the steps of reacting a body-fluid sample with said peptide(s), and detecting antibodies in the sample that are immunoreactive with each peptide. It is preferred that the assay be quantitative and accordingly be used to compare the level of each antibody in order to determine the relative magnitude of the immune response to each peptide.

The methods of the invention are generally applicable to immunoassays, such as enzyme linked immunosorbent assay (ELISAs), radioimmunoassay (RIA), immunoprecipitation, Western blot, dot blotting, FACS analyses and other methods known in the art.

In one preferred embodiment, the immunoassay includes a peptide antigen inmmobilized on a solid support, e.g., an ELISA assay. It will be appreciated that the immunoassay may be readily adapted to a kit format exemplified by a kit which comprises: (A) one or more peptides of the invention bound to a solid support; (B) a means for collecting a sample from a subject; and (C) a reaction vessel in which the assay is carried out. The kit may also comprise labeling means, indicator reaction enzymes and substrates, and any solutions, buffers or other ingredients necessary for the immunoassay.

Diagnosis of Influenza Infection

The present invention is also directed to diagnosis of an influenza infection. General methods for diagnosis of an influenza infection are well known to a skilled artisan and are disclosed for instance in U.S. Pat. No. 6,811,971. The present invention provides a method of identifying influenza virus in a biological sample by (a) contacting the biological sample with a nucleic acid primers amplifying the part of virus genome encoding for the divalent sialoside binding site of the X31-hemagglutinin protein as disclosed below under conditions allowing polymerase chain reaction; and (b) determining the sequence of the amplified nucleic acid in the biological sample, to thereby identify the presence and type of influenza virus. Alternatively, the presence of influenza virus can be detected by (a) contacting the biological sample with an antibody or antibody fragment specifically recognizing the divalent sialoside binding site of the X31-hemagglutinin protein as disclosed below; and (b) detecting immunocomplexes including said antibody or antibody fragment in the biological sample, to thereby identify the presence and type of influenza virus in the biological sample.

The large polylactosamine epitopes: high affinity ligands for influenza virus The present invention is directed to a peptide epitoes of hemagglutinin protein of influenza virus derived from the high affinity binding site for sialylated ligands The inventors have prevoisly found out that the influenza virus hemagglutinin bind complex human glycans such as poly-N-acetyllactosamine type carbohydrates using a large binding site according to the invention on its surface, WO2005/037187. The present invention is especially directed to special short peptide epitopes and combinations thereof derived from the large binding site. The special large poly-N-acetyllactosamines are called here “the large polylactosamine epitopes”.

The Large Binding Site

Furthermore, the present invention is especially directed to the novel large binding site on surface of hemagglutinin, called here “the large binding site”. The large binding site binds effectively special large polylactosmine type structures and analogs and derivatives thereof with similar binding interactions and/or binding surface in the large binding site. The large binding site includes:
1. the known primary binding site for sialylated structures in human influenza hemagglutinin, the region of the large binding site is called here “the primary site” or “Region A” and
2. so called secondary sialic acid binding site on the surface of the hemagglutinin, wherein the sialic acid or surprisingly also certain other terminal monosaccharide residues or analogs thereof can be bound by novel binding mode, the region of the large binding site is called here “the secondary site” or “Region C” and
3. a groove-like region on surface of hemagglutinin bridging the primary and secondary sites, called here “the bridging site” or “Region B”.

The Conserved Peptide Sequences of the Large Binding Site

Molecular modelling of mutated sites on the surface of influenza hemagglutinin revealed that many of amino acid residues on the large binding site are strongly conserved and part of the amino acid residues are semiconservatively conserved. The conservation of the protein structures further indicates the biological importance of the large binding site of the hemagglutinin. The virus cannot mutate nonconservatively the large binding site without losing its binding to the sialylated saccharide receptors on the target tissue. It clear that the large binding site is of special interest in design of novel medicines for influenza, which can stop the spreading of the virus.

Conservation of the Large Binding Site Between Species

Furthermore, it was found out that the large binding sites in general are conserved between various influenza virus strains. Mutations were mapped from hemagglutinins from 100 strains closely related to strain X31. The large binding site was devoid of mutations or contained conservatively mutated amino acids in contrast to the surrounding regions. The large binding site recognized sialylated polylactosamines.

Animal hemagglutinins, especially avian hemagglutinins, are important because pandemic influenza strains has been known to have developed from animal hemagglutinins such as hemagglutinins from chicken or ducks. Also pigs are considered to have been involved in development of new influenza strains. The recognition of large carbohydrate structures on the surface of influenza hemagglutinin has allowed the evolution of the large binding site between terminal carbohydrate structures containing α3- and/or α6-linked sialic acids. The pandemic strains of bird origin may be more α3-sialic acid specific, while the current human binding strains are more α6-specific. The present invention is further directed to mainly or partially α3-specific large binding sites. The present invention is further directed to substances to block the binding to mainly or partially α6-specific large binding sites.

Design of Vaccines and Antibodies.

The large binding site and its conserved peptide sequences are of special interest in design of novel vaccines against influenza virus. The general problem with vaccines against influenza is that the virus mutates to immunity. A vaccine inducing the production of antibodies specific for the large binding site and its conserved peptide sequences will give general protection against various strains of influenza virus.

Furthermore, the invention is directed to the use of antibodies for blocking binding to the large binding site. Production of specific antibodies and human or humanized antibodies is known in the art. The antibodies, especially human or humanized antibodies, binding to the large binding site, are especially preferred for general treatment of influenza in human and analogously in animal.

Methods for producing peptide vaccines against influenza virus are well-known in the art. The present invention is specifically directed to selecting peptide epitopes for immunization and developing peptide vaccines comprising at least one di- to decapeptide epitope, more preferably at least one tri- to hexapaptide epitope, and even more preferably at least one tri to pentapeptide epitope of the “large binding site” described by the invention..

The peptide epitopes are preferably selected to contain the said peptide from among the important binding and/or conserved amino acids according to the invention and as described in the previous influenza applications of the inventors, more preferably at least one peptide epitope is selected from region B. In another preferred embodiment two peptides are selected for immunization with two peptides so that at least one is from region B and one from region A or B. Preferably the peptide epitope is selected to comprise at least two conserved amino acid residues, in another preferred embodiments the peptide epitope is selected to comprise at least three conserved amino acid residues. In a preferred embodiment peptide epitope is modelled to be well accessible on the surface of the hemagglutinin protein.

Combinations of Peptide Epitopes

It was realized that single peptide epitope has multiple strain specific variants. It would be useful to use several variants for current virus type for diagnostic and therapeutics according to the invention. The invention is especially directed to the use of the natural peptide sequences derived from the hemagglutinins, e.g ones demonstrated in the Figures. The invention is further directed to use of multiple epitopes from different regions of the hemagglutinin large binding site in order to provide maximal immune recognition of virus by patients with different immune history against the viruses and different immune system, this was demonstrated with ELISA assay measuring varying reactions from several persons.

Those of skill in the art will realize that association of natural ligands or substrates with the binding pockets of their corresponding receptors or enzymes is the basis of many biological mechanisms of action. The term “binding site”, as used herein, refers to a region of a molecule or molecular complex, that, as a result of its shape, favorably associates with another chemical entity or compound. Similarly, many drugs exert their biological effects through association with the binding pockets of receptors and enzymes. Such associations may occur with all or any parts of the binding pockets. An understanding of such associations will help lead to the design of drugs having more favorable associations with their target receptor or enzyme, and thus, improved biological effects. Therefore, this information is valuable in designing potential ligands or inhibitors of receptors or enzymes, such as blockers of hemagglutinin.

Molecular Modelling Techniques

Molecular modelling techniques can be applied to the atomic co-ordinates of the large binding site of influenza hemagglutinin to derive a range of 3D models and to investigate the structure of ligand binding sites. A variety of molecular modelling methods are available to the skilled person for use according to the invention [e.g. ref. 5].

At the simplest level, visual inspection of a computer model of the large binding site of influenza hemagglutinin can be used, in association with manual docking of models of functional groups into its binding sites.

Software for implementing molecular modelling techniques may also be used. Typical suites of software include CERIUS2 [Available from Molecular Simulations Inc], SYBYL [Available from Tripos Inc], AMBER [Available from Oxford Molecular], HYPERCHEM [Available from Hypercube Inc], INSIGHT II [Available from Molecular Simulations Inc], CATALYST [Available from Molecular Simulations Inc], CHEMSITE [Available from Pyramid Learning], QUANTA [Available from Molecular Simulations Inc]. These packages implement many different algorithms that may be used according to the invention (e.g. CHARMm molecular mechanics [Brooks et al. (1983) J. Comp. Chem. 4: 187-217]). Their uses in the methods of the invention include, but are not limited to: (a) interactive modelling of the structure with concurrent geometry optimisation (e.g. QUANTA); (b) molecular dynamics simulation of the large binding site of influenza hemagglutinin (e.g. CHARMM, AMBER); (c) normal mode dynamics simulation of the large binding site of influenza hemagglutinin (e.g. CHARMM).

Modelling may include one or more steps of energy minimisation with standard molecular mechanics force fields, such as those used in CHARMM and AMBER.

These molecular modelling techniques allow the construction of structural models that can be used for in silico drug design and modelling.

Pharmacophore Searching

As well as using de novo design, a pharmacophore of the large binding site of influenza hemagglutinin can be defined i.e. a collection of chemical features and 3D constraints that expresses specific characteristics responsible for biological activity. The pharmacophore preferably includes surface-accessible features, more preferably including hydrogen bond donors and acceptors, charged/ionisable groups, and/or hydrophobic patches. These may be weighted depending on their relative importance in conferring activity.

Pharmacophores can be determined using software such as CATALYST (including HypoGen or HipHop) [Available from Molecular Simulations Inc], CERIUS2, or constructed by hand from a known conformation of a lead compound. The pharmacophore can be used to screen in silico compound libraries, using a program such as CATALYST [Available from Molecular Simulations Inc].

Suitable in silico libraries include the Available Chemical Directory (MDL Inc), the Derwent World Drug Index (WDI), BioByteMasterFile, the National Cancer Institute database (NCI), and the Maybridge catalog.

The term “treatment” used herein relates both to treatment in order to cure or alleviate a disease or a condition, and to treatment in order to prevent the development of a disease or a condition. The treatment may be either performed in an acute or in a chronic way.

The pharmaceutical composition according to the invention may also comprise other substances, such as an inert vehicle, or pharmaceutically acceptable carriers, preservatives etc., which are well known to persons skilled in the art.

The substance or pharmaceutical composition according to the invention may be administered in any suitable way, although an oral or nasal administration especially in the form of a spray or inhalation are preferred. The nasal and oral inhalation and spray dosage technologies are well-known in the art. The preferred dose depend on the substance and the infecting virus. In general dosages between 0.01 mg and 500 mg are preferred, more preferably the dose is between 0.1 mg and 50 mg. The dose is preferably administered at least once daily, more preferably twice per day and most preferably three or four times a day. In case of excessive secretion of mucus and sneezing or cough the dosage may be increased with 1-3 doses a day.

The present invention is directed to novel divalent molecules as substances. Preferred substances includes preferred molecules comprising the flexible spacer structures and peptide and/or oxime linkages. The present invention is further directed to the novel uses of the molecules as medicines. The present invention is further directed to in methods of treatments applying the substances according to the invention.

The term “patient”, as used herein, relates to any human or non-human mammal in need of treatment according to the invention.

Glycolipid and carbohydrate nomenclature is according to recommendations by the IUPAC-IUB Commission on Biochemical Nomenclature (Carbohydrate Res. 1998, 312, 167; Carbohydrate Res. 1997, 297, 1; Eur. J. Biochem. 1998, 257, 29).

It is assumed that Gal, Glc, GlcNAc, and Neu5Ac are of the D-configuration, Fuc of the L-configuration, and all the monosaccharide units in the pyranose form. Glucosamine is referred as GlcN or GlcNH₂ and galactosamine as GalN or GalNH₂. Glycosidic linkages are shown partly in shorter and partly in longer nomenclature, the linkages of the Neu5Ac-residues α3 and α6 mean the same as α2-3 and α2-6, respectively, and with other monosaccharide residues α1-3, β1-3, β1-4, and β1-6 can be shortened as α3, β3, β4, and β6, respectively. Lactosamine refers to N-acetyllactosamine, Galβ4GlcNAc, and sialic acid is N-acetylneuraminic acid (Neu5Ac, NeuNAc or NeuAc) or N-glycolylneuraminic acid (Neu5Gc) or any other natural sialic acid. Term glycan means here broadly oligosaccharide or polysaccharide chains present in human or animal glycoconjugates, especially on glycolipids or glycoproteins. In the shorthand nomenclature for fatty acids and bases, the number before the colon refers to the carbon chain length and the number after the colon gives the total number of double bonds in the hydrocarbon chain.

Antibody Substances

Every method of using antibody substances of the invention, whether for therapeutic, diagnostic, or research purposes, is another aspect of the invention. For example, the invention further contemplates use of the peptide motifs as a method for screening for antibody substances. One aspect the invention provides a method of screening an antibody substance for peptide motif or peptide motifs and influenza virus neutralization activity comprising: contacting a peptide motif/antigen and influenza virus in the presence and absence of an antibody substance; and measuring binding between the peptide motif/antigen and the virus in the presence and absence of the antibody substance, wherein reduced binding in the presence of the antibody substance indicates virus neutralization activity for the antibody substance; wherein the peptide motif/antigen comprises at least one member selected from the group consisting of KVR, KVN, WVR, TPNPENGT, KANPANDL, VTKGVSAS, GGSNA, and EASSGVSSA region; and combinations thereof; wherein the virus is at least one member selected from the group consisting of H1, H2, H3, H4 or H5 HA subtype of the influenza virus A; and wherein the antibody substance comprises an antibody substance according to the invention.

For example, one aspect of the invention is a method for inhibiting, preventing or alleviating influenza virus caused symptoms, by vaccination, comprising administering to a mammalian subject in need of inhibition, prevention or alleviation of influenza virus caused symptoms a peptide motif or peptide motifs according to the invention, in an amount effective to inhibit, alleviate or prevent influenza virus caused symptoms. Methods to determine the extent of inhibition, prevention and alleviation influenza virus caused symptoms are described herein.

For example, one aspect of the invention is a method for inhibiting, preventing or alleviating influenza virus caused symptoms comprising administering to a mammalian subject in need of inhibition, prevention or alleviation of influenza virus caused symptoms an antibody substance according to the invention, in an amount effective to inhibit, alleviate or prevent influenza virus caused symptoms. Methods to determine the extent of inhibition, prevention and alleviation influenza virus caused symptoms are described herein.

The invention further provides a method of inhibiting, preventing or alleviating influenza virus caused symptoms comprising steps of: (a) determining peptide motifs and/or region composition of an influenza virus from a sample or a mammalian subject, (b) assaying binding between peptide motifs and the antibody substances; and (c) administering to a subject an antibody substance according to the invention, wherein the antibody substance binds to peptide motif(s) identified in step (a).

The invention further provides a method of inhibiting, preventing or alleviating influenza virus caused symptoms comprising steps of: (a) determining peptide motifs and/or region composition of an influenza virus from a sample or a mammalian subject, (b) administering to a subject peptide motif(s) according to the invention.

Antibody substances of the invention are useful for preventing, alleviating and/or inhibiting influenza causes symptoms. The invention provides antibody substances for administration to human beings (e.g., monoclonal and polyclonal antibodies, single chain antibodies, chimeric antibodies, bifunctional/bispecific antibodies, humanized antibodies, human antibodies, and complementarity determining region (CDR)-grafted antibodies, including compounds which include CDR sequences which specifically recognize a polypeptide of the invention) specific for polypeptides of interest to the invention. Preferred antibodies are human antibodies which are produced and identified according to methods described in WO 93/11236, published Jun. 20, 1993, which is incorporated herein by reference in its entirety. Antibody fragments, including Fab, Fab′, F(ab′)2, Fv, and single chain antibodies (scFv) are also provided by the invention. Various procedures known in the art may be used for the production of polyclonal antibodies to peptide motifs and regions or fragments thereof. For the production of antibodies, any suitable host animal (including but not limited to rabbits, mice, rats, or hamsters) are immunized by injection with a peptide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG {Bacille Calmette-Guerin) and Corγnebacterium parvum.

A monoclonal antibody to a peptide motif(s) may be prepared by using any technique which provides for the production of antibody molecules by continuous cell lines in culture. These include but are not limited to the hybridoma technique originally described by Kδhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XXO BuI; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sd 81: 6851-6855, 1984; Neuberger et al, Nature 312: 604-608, 1984; Takeda et al, Nature 314: 452-454; 1985). Alternatively, techniques described for the production of single- chain antibodies (U.S. Pat. No. 4,946,778) can be adapted to produce influenza- specific single chain antibodies.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab')2 fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2nd Edition, Garland Publishing, New York, 1996). The CDR regions in any given antibody may be found within several amino acids of these approximated residues set forth above. An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity determining regions of a monoclonal antibody secreted by a hybridoma are also contemplated. Using the conserved framework sequences surrounding the CDRs, PCR primers complementary to these consensus sequences are generated to amplify a CDR sequence located between the primer regions. Techniques for cloning and expressing nucleotide and polypeptide sequences are well-established in the art [see e.g., Sambrook et al., Molecular Cloning: A Laboratory Manual, 2nd Edition, Cold Spring Harbor, New York (1989)]. The amplified CDR sequences are ligated into an appropriate plasmid. The plasmid comprising one, two, three, four, five and/or six cloned CDRs optionally contains additional polypeptide encoding regions linked to the CDR.

Nucleic Acids of the Invention

RNA viruses, including the influenza A virus, tend to have high mutation rates due to the low fidelity nature of RNA replication when compared to DNA replication. As a result, influenza viruses tend to evolve rapidly. Furthermore, influenza A viruses tend to undergo genetic reassortment between viral strains, which mechanism has contributed to the development of the various HA and NA subtypes. The inventors compared the sequence of the hemagglutinin (“HA”) gene from known influenza A sequences. Surprisingly, despite the high mutation rate within influenza viruses, the inventors have discovered short regions of highly conserved sequences unique to all subtypes, which regions are suitable to identify or detect the presence of influenza A and/or a subtypes or subtypes in a sample.

The sequences used in the comparison were obtained from publicly available databases and were compared using a variety of sequence comparison software Influenza Virus Resource.

These sequence comparisons allowed the inventors to develop forward and reverse primers set out in WO 2008/049974 by the same inventors, directed to conserved regions of the HA gene of influenza virus subtypes H1, H3 and H5, for use in a detection assay, for example, reverse-transcription followed by polymerase chain reaction amplification (“RT-PCR”). The comparison of such a large number of viruses allowed for the design of primers directed to well-conserved regions of the HA gene, thus targeting regions that are less likely to be affected by mutational changes and thereby providing primers that can detect a larger pool of H variants than primers that are currently available.

The term “isolate” as used herein refers to a particular virus or clonal population of virus particles, isolated from a particular biological source, such as a patient, which has a particular genetic sequence. Different isolates may vary at only one or several nucleotides, and may still fall within the same viral subtype. A viral subtype refers to any of the subtypes of HA classified according to the antigenicity of these glycoproteins.

The inventors found that in certain conserved regions, one or more nucleotides at a specific location vary between isolates. For those regions, a family of primers can be developed, each primer within the family being based on a conserved sequence of the HA gene, but varying at one or more particular bases within the conserved sequence.

As will be understood by a skilled person, a “primer” is a single-stranded DNA or RNA molecule of defined sequence that can base pair to a second DNA or RNA molecule that contains a complementary sequence (the target). The stability of the resulting hybrid molecule depends upon the extent of the base pairing that occurs, and is affected by parameters such as the degree of complementarity between the primer and target molecule and the degree of stringency of the hybridization conditions. The degree of hybridization stringency is affected by parameters such as the temperature, salt concentration, and concentration of organic molecules, such as formamide, and may be determined using methods that are known to those skilled in the art. Primers can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), nucleic acid microarrays, and other methods that are known to those skilled in the art.

The term “RNA” refers to a sequence of two or more covalently bonded, naturally occurring or modified ribonucleotides. The RNA may be single stranded or double stranded. The term “DNA” refers to a sequence of two or more covalently bonded, naturally occurring or modified deoxyribonucleotides, including cDNA and synthetic (e.g., chemically synthesized) DNA, and may be double stranded or single stranded. By “reverse transcribed DNA” or “DNA reverse transcribed from” is meant complementary or copy DNA (cDNA) produced from an RNA template by the action of RNA-dependent DNA polymerase (reverse transcriptase).

Influenza A virus is a single stranded RNA virus and in some embodiments, the primer has a DNA sequence that corresponds to the RNA sequence of a conserved region of the HA gene of human, avian and/or swine influenza virus subtype H1-5. Such primers may be used as a forward primer when sequencing or amplifying DNA reverse transcribed from the HA genes.

Furthermore, a skilled person will understand that, although the primers are based on conserved sequences, one or more bases within the conserved sequences can be substituted, inserted or deleted, provided that the mutated primer will still hybridize with the target sequence in a sample with the same or similar stringency as the original primer sequence. Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, stringent conditions are selected to be about 50 C lower than the thermal melting point for the specific sequence at a defined ionic strength and pH.

A skilled person will understand that having multiple substitution mutations in a short sequence will decrease the strength of hybridization of the primer to the complement of the original, unmutated primer, and that the spacing and location of the mutations within the primer sequence will also affect the strength or stringency of hybridization. Furthermore, a skilled person will understand that insertion or deletion of one or more nucleotides in a short sequence will also decrease the strength of hybridization of the primer to the complement of the original, unmutated primer, and that having insertions or deletions of one or more nucleotides in more than one location in a short sequence may significantly alter the hybridization of the primer to the complement of the unmutated sequence.

In some embodiments, the primer may be modified with a label to allow for detection of the primer or a DNA product synthesized or extended from the primer. For example, the label may be a fluorescent label, a chemiluminescent label, a coloured dye label, a radioactive label, a radiopaque label, a protein including an enzyme, a peptide or a ligand for example biotin.

Skilled artisan also understands that primers can surround at least one peptide epitope of the present invention, e.g. peptide 1 region (prepeptide 1, peptide1 and/or postpeptide1), or at least two regions, e.g. peptide 1 and peptide 2 and their surrounding regions. Alternatively, two peptide regions can encompass peptides 2 and 4, or 4 and 3. In the other words, primers can be in between peptide sequences. Furthermore, primers can encompass at least three peptide regions, e.g. peptide 1, 2 and 4, or 2, 4 and 3. One embodiment favors primers which bind upstream of peptide 1 and downstream of peptide 3, i.e. encompassing the whole large binding region. This region is about 500-520 nucleotides and resulting fragment can be about 500, 510, 520, 530, 540, 550, 560 or 570 by of length. Alternatively, is some applications about 600, 700, 800 or longer by fragments are desired.

Skilled artisan also understands that a primer sequence may be located in between peptide epitopes or motifs.

Peptide region is defined as a amino acid sequence which encompasses conserved tri- or oligopeptide motifs described herein. For example, conserved peptide motif of peptide 3 of H1 is KVR. Peptide region of KVR means amino acid sequences upstream (toward amino terminus) of KVR and downstream (toward COOH terminus) of KVR, usually 1, 2, 3, 4 or 5 amino acids in length. The upstream and downstream amino acid sequences can also be 6, 7, 8, 9, 10 or longer. These pre and post peptide sequences can be conserved and the invention is directed to identify these conserved sequences. The pre and post peptide sequences can also contain non conserved amino acids which are depicted as X and can be any amino acid or limited to few amino acids which are seen to vary in or between HA gene.

Peptide region also contemplates corresponding nucleotide sequences encoding the amino acids in the region or epitope. Due to degeneracy many nucleotide sequences can encode a single amino acid and are also included in the present invention.

The present invention is directed to all influenza virus A regardless of host species. Host species can be avian, swine, or mammalian. Preferred avian host consist of chicken, duck, and quail. Preferred feline species consist of cat, tiger and leopard. Other preferred mammalians are dog, equine, mouse, seal, whale and mink. Most preferred mammalian is human. Most preferred host is human. Other species include camel Skilled artisan understands that all influenza A types which infect host species other than human may potentially mutate and infect humans. Therefore the present invention is suitable for screening and anticipating peptide antibodies which are to be administered to humans to treat influenza, alleviate influenza symptoms, to treat and/or alleviate symptoms caused by influenza conditions, for example, secondary infection caused by bacteria. Most preferred is the prevention of influenza symptoms by determining peptide epitopes of an influenza type and administering peptides of the present invention.

Skilled artisan understands to screen for other peptide epitope encompassing and encoding primers using methodology described herein. HA subtypes for additional H1, H3 and H5 can be screened using methodology. Preferably, other HA subtypes like H2, H6-17 can be screened to anticipate their potential threat to mutate and acquire human-to-human or animal-to-human transmission.

The invention is well suited for preventing influenza in a patient. HA subtype is determined using primers of the present invention and peptide epitopes of the present invention are administered into a patient, and immune defense is raised against peptides, thus, against influenza virus.

The amino acid sequence and 3D-structure of influenza X-31 hemagglutinin is described previously, e.g., in PCT/F12006/050157 (published as WO2006111616).

### EXAMPLES

### Example 1

### Modeling Studies of the Influenza Hemagglutinin

Introduction—The X-ray crystallographic structure of the hemagglutinin of the X-31 strain of human influenza virus was used for the docking (PDB-database, www.rcsb.org/pdp, the database structure 1HGE). The structure used in the modelling is a complex structure including Neu5Acα-OMe at the primary sialic acid binding site, the large oligosaccharide modelled to the site had one Neu5Acα-superimposable to the one in the 1HGE, but glycosidic glycan instead of the methyl group. The studies and sequence analyses described below in conjunction with hemagglutination-inhibition studies used for evaluation of the binding efficacy of the different branched poly-N-acetylactosamine inhibitors.

In addition to the primary site, which binds to both sialyl-α3-lactose and sialyl-α6-lactose, a secondary site exists which has been previously found to bind sialyl-α3-lactose as well but not sialyl-α6-lactose. The present conformational peptide 3 and additional peptides 1 and 2 were modelled to the surface of hemagglutinin in the carbohydrate binding site, peptide 3 especially as a protruding loop structure.

### Example 2

### Assays With 2 Immobilization Chemistries

Materials and Methods for ELISA Assays of Peptides

ELISA Assays on Maleimide-Activated Plates

Peptides containing cysteine were bound through the cysteine sulfhydryl group to maleimide activated plates (Reacti-Bind™ Maleimide activated plates, Pierce). The peptides sequences were as follows:
Biotin-aminohexanoyl-SYACKR (custom product, CSS, Edinburgh, Scotland)
Biotin-aminohexanoyl-SKAYSNC (custom product, CSS, Edinburgh, Scotland)
CYPYDVPDYA (HA11; Nordic Biosite)

All peptides were dissolved in 10 mM sodium phosphate/0.15 M NaCl/2 mM EDTA, pH 7.2, to a concentration of 5 nmol/ml. One hundred microliters of the peptide solution (0.5 nmol of peptide) was added to each well and allowed to react overnight at +4° C. The plate was then washed three times with 10 mM sodium phosphate/0.15 M NaCl/0.05% Tween-20, pH 7.2).

The unreacted maleimide groups were blocked with 2-mercaptoethanol: 150 μl of 1 mM 2-mercaptoethanol in 10 mM sodium phosphate/0.15 M NaCl/2 mM EDTA, pH 7.2 was added to each well and allowed to react for 1 hour at RT. The plate was then washed three times with 10 mM sodium phosphate/0.15 M NaCl/0.05% Tween-20, pH 7.2. The plate was further blocked with 1% bovine serum albumin (BSA) in 10 mM sodium phosphate/0.15 M NaCl/0.05% Tween-20, pH 7.2, and then washed with 10 mM sodium phosphate/0.15 M NaCl/0.05% Tween-20/0.2% BSA, pH 7.2 (washing buffer).

Serum was obtained from six healthy individuals (29-44 years of age), and dilutions 1:10, 1:100 and 1:1000 were prepared from all but one serum sample in the washing buffer. The serum obtained from person nr. 5 was instead diluted 1:25, 1:250 and 1:2500 in the washing buffer. One hundred microliters of each serum sample was added to the wells and incubated for 30 mins at RT. Control wells contained no peptide but both 2-mercaptoethanol and BSA blockings were employed. All incubations were performed in duplicates.

The plate was then washed with the washing buffer 8 times with at least 5 min incubation period between change of the washing liquid.

The bound serum antibodies were quantitated by adding anti-human IgG (rabbit)—HRP conjugate (Sigma) in 1:30000 dilution to each well. After one hour incubation at RT, the plate was washed five times with the washing buffer. One hundred microliters of TMB+ color reagent (Dako Cytomation) was then added. The absorbance was read at 650 nm after 15 mins. Immediately after this measurement 100 μl of 1 M sulphuric acid was added and the absorbance read at 450 nm. Results are shown in Table 1.

ELISA Assays on Streptavidin-Coated Plates

Biotinylated peptides were bound to streptavidin-coated plates (Pierce). The peptides sequences were as follows:
Biotin-aminohexanoyl-PWVRGV (custom product, CSS, Edinburgh, Scotland)
Biotin-aminohexanoyl-SYACKR (custom product, CSS, Edinburgh, Scotland)
Biotin-aminohexanoyl-SKAYSNC (custom product, CSS, Edinburgh, Scotland)

Prior to peptide immobilization, plates were blocked with 1500 of 0.5% BSA in 10 mM sodium phosphate/0.15 M NaCl/0.05% Tween-20, pH 7.2, for 1.5 h at RT. The plate was then washed three times with 10 mM sodium phosphate/0.15 M NaCl/0.05% Tween-20, pH 7.2.

Peptides were dissolved in 10 mM sodium phosphate/0.15 M NaCl, pH 7.2, to a concentration of 0.5 nmol/ml. One hundred microliters of the peptide solutions (50 pmol of the peptide) were added to the wells and allowed to react overnight at +4° C. The plates were then washed four times with 10 mM sodium phosphate/0.15 M NaCl/0.05% Tween-20/0.2% BSA, pH 7.2 (washing buffer).

Serum was obtained from six healthy individuals (29-44 years of age), and dilutions 1:10, 1:100 and 1:1000 were prepared from all but one serum sample in the washing buffer. The serum obtained from person nr. 5 was instead diluted 1:25, 1:250 and 1:2500 in the washing buffer. One hundred microliters of each serum sample was added to the wells and incubated for 60 mins at RT. Control wells did not contain peptides but were blocked as above. All incubations were performed in duplicates.

After serum incubation the plate was washed with the washing buffer 8 times with at least 5 min incubation period between change of the washing liquid.

The bound serum antibodies were quantitated by adding anti-human IgG (rabbit)—HRP conjugate (Sigma) in 1:30000 dilution to each well. After one hour incubation at RT, the plate was washed five times with the washing buffer. One hundred microliters of TMB+ color reagent (Dako Cytomation) was then added. The absorbance was read at 650 nm after 15 mins. Immediately after this measurement 100 μl of 1 M sulphuric acid was added and the absorbance read at 450 nm.

Results of ELISA Assays of Antigen Peptides

Design of the Experiments

Three antigen peptides were analysed against natural human antibodies from healthy adults. The individuals were selected based on the resistance against influenza for several years. The persons had been in close contact with persons with distinct influenza type disease in their families and/or at work but have not been infected for several years. At the time of blood testing two of the persons had influenza type disease at home but persons were suffering from only mild disease. The persons were considered to have good immune defense against current influenza strains.

The antigen peptides were selected to correspond structures present on recent influenza A (H3N2) strains in Finland (home country of the test persons). The assumption was that the persons had been exposed to this type of viruses and they would have antibodies against the peptides, in case the peptides would be as short linear epitopes effectively recognizable by human antibodies and peptide epitopes would be antigenic in human. The invention revealed natural human antibodies against each of the peptides studied. The data indicates that the peptides are antigenic and natural antibodies can recognize effectively such short peptide epitopes.

All antigen peptides 1-3 were tested as N-terminal biotin-spacer conjugates, which were immobilized on a streptavidin plate. Aminohexanoic acid spacer was used to allow recognition of the peptides without steric hindrance from protein. It is realized that the movement of the N-terminal part of peptide was limited, which would give conformational rigidity to the peptide partially mimicking the presence on a polypeptide chain.

The Peptides 1 and 2 Were Also Tested on Maleimide Coated Plates.

The peptide 1 (Biotin-aminohexanoic-SKAYSNC) was also tested as conjugated from natural C-terminal Cys-residue in a antigen peptide, the peptide further contained spacer-biotin structure at amino terminal end of the peptide. The peptide presented natural C-terminal and Cys-linked presentation at C-terminus of the peptide presenting a preferred conformational structure. The presentation as natural like epitope was further supported by spacer structure blocking the N-terminus and restricting its mobility.

The peptide 2 (Biotin-aminohexanoic-SYACKR) was also tested as conjugated from natural Cys-residue in the middle of the antigen peptide. The peptide presented natural middle Cys-linked presentation at C-terminus of the peptide presenting a preferred conformational structure. The presentation as natural like epitope was further supported by spacer structure blocking the N-terminus and restricting its mobility.

Control and Core Peptide

A commercial peptide CYPYDVPDYA (HA11-peptide), which has been used as a recognition tag on recombinant proteins was used as a control and for testing of analysis of binding between a free core peptide and human antibodies. Due to restricted availability of at least N-terminal sequence the peptide would not be very effective in immunization against the viral as therapy. This peptide is known to be antigenic in animals under immunization conditions and antibodies including polyclonals from rabbit, mice etc. The ELISA assay was controlled by effective binding of commercial polyclonal antibody from rabbit to the peptide coated on a maleimide plate, while neglicible binding was observed without the peptide.

Results

The absorbance was recorded by two methods (A450 and A650) and with three different dilutions giving similar results (the results with optimal dilutions giving absorbance values about 0.1 AU to about 0.8 AU and by absorbance at 450 nm are shown).

Peptide 1 as Aminoterminal Conjugate and C-Terminal Cys-Conjugate

Biotin-aminohexanoic-SKAYSNC was tested against the 6 sera as N-terminal conjugate on a streptavidin plate. The sera 3 and 4 showed strongest immune response before serum 2, while sera 1, 5 and 6 were weakly or non-reactive against the construct.

The C-terminal cysteine conjugate of peptide 1 reacted with sera in the order from strongest to weaker: 6, 3, 4, and 2, while 1 and 5 were weakly or non-reactive against the construct. The results indicated, that both conjugates reacted remarkably similarily with antibodies except the serum 6 which contained antibodies preferring the structure including the immobilized cysteine as in natural peptides on viral surface.

Peptide 2 as Aminoterminal Conjugate and Middle Cys-Conjugate

Biotin-aminohexanoic-SYACKR was tested against the 6 sera as N-terminal conjugate on a streptavidin plate. The sera 2 and 5 showed strongest immune response before sera 3, 4 and 6, while serum 1 showed weakest reaction.

The middle cysteine conjugate of peptide 2 reacted with sera similarily but reactions with serum 5 was weaker and the serum 6 showed the strongest reponse, see Table 1. The results indicated, that both conjugates reacted remarkably similarily with antibodies except the serum 6 which contained antibodies preferring the structure including the immobilized cysteine as in natural peptides on viral surface.

Peptide 3

Peptide 3 has distinct pattern of immune recognition as shown in Table 1.

Correlation of the Immune Reaction With Viral Presentation of the Peptides 1-3 and HA11

More than hundred recently cloned human influenza A viruses were studied with regard to presentation of peptides 1-3. It was realized that there is one to a few relatively common escape mutants of each one of these, which would be different in antigenicity in comparision to the peptides 1-3. The analysis further reveled that on average the viruses contain two of the peptides 1-3. Thus the result that each influenza resistant test subject had antiserum at least against two of peptides fits well data about the recent viruses in Finland. The data further support the invention about combination of the antigenic peptides. The combination of at least two peptides is preferred.

The control core sequence HA11 is present as very conserved sequence in most influenza A viruses and thus all persons would have been immunized against it as shown by the results in Table 1.

### Example 4

Multiple alignment of amino acid sequences from various HA subtypes and hosts.

Altogether 158 sequences and 788 sequences were used for the analysis. In some cases all peptide sequences of a subtype were aligned in groups of 200-400 sequences. The sequences were aligned using Influenza Virus Resource alignment tools and the variant amino acids were visually observed within the peptide regions of the invention.

Comparisons were also made within an HA subtype by aligning each HA subtypes and observing variation in the peptide regions of the invention.

### Example 5

### Analysis of Current Influenza Peptides Including Cyclic Forms of Peptides 3

Linear and cyclic peptides from recent influenza H1 and H3 viruses were tested for binding to antibodies from serum of 8 persons similarily as in ELISA assay as in Example 2. The process was optimized increasing washing the plates. The assay revealed strong immune individual specific responses against all tested peptides. This is partially expected to be based on the infections of person by older viruses or more current H1 and/or H3 viruses with current sequences

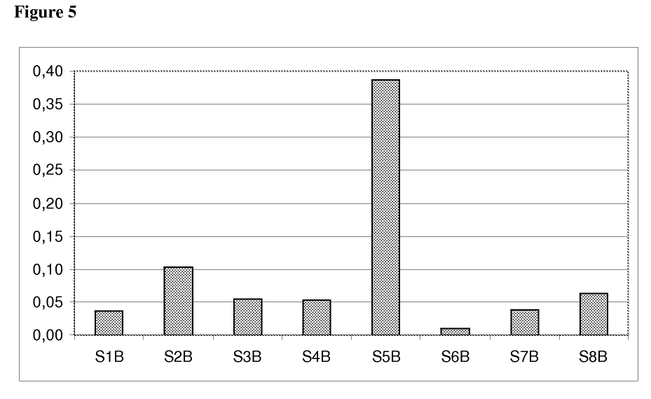

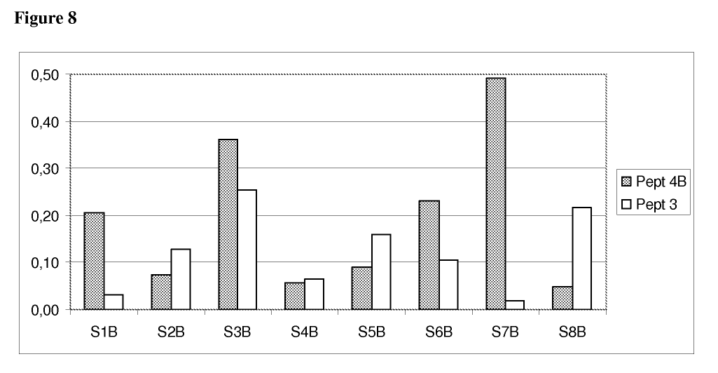

The assays revealed especially that cyclic peptides 3 in cyclic form are especially strong immugens/antibody targets. FIG. 8 shows that cyclic Peptide 4b bind generally more strongly antibodies than the corresponding linear peptide 3 analyzed again (also used in Example 2). Also the H1 peptide 3 in cyclic form showed unusually high response, especially with a person S5B, FIG. 5, who had been vaccinated against influenza (vaccines comprise regularily both H1 and H3 virus though the infection with H1 may be otherwise more rare). This indicates that the binding of conformational structure 3 is especially useful. It is realized that in Example 2 the differences in maleimide linked epitope linking conformationally from cysteine and the N-terminally linked structures from biotin indicates that the cystein linkage would provide beneficial conformational peptide for certain natural anti-influenza antibodies.

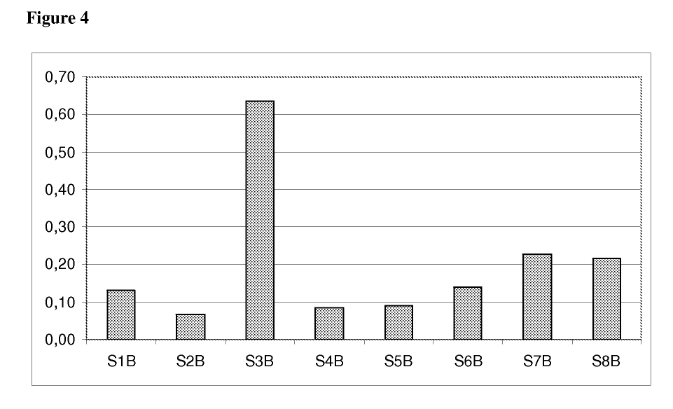

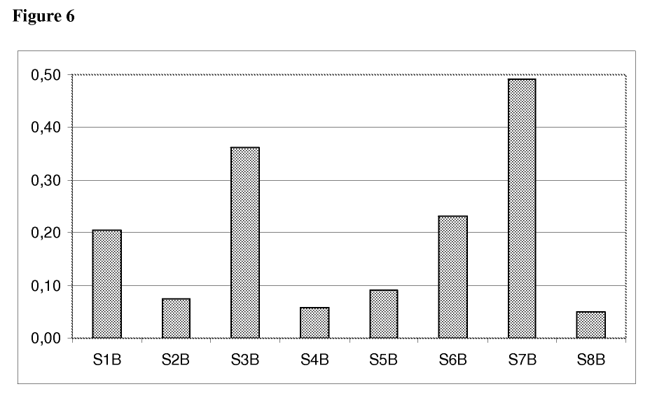

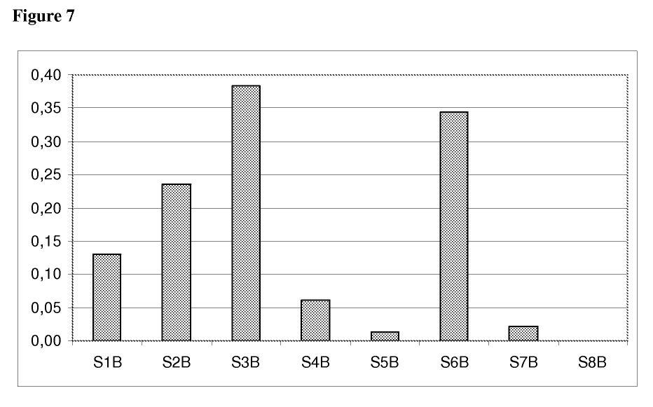

It is thus realized that the novel peptides are useful in recognition of influenza immune reactions in context of vaccination with whole viruses or larger hemagglutinin peptides or proteins, person S5B FIG. 5. It is further realized and preferred that immunoassays directed to measuring the antibodies against influenza are especially useful for diagnosis of influenza and even specific type of influenza with regard to hemagglutinin structures. At least persons S3B (required hospital visit) and S7B were considered as recently infected quite severely with influenza and showed strong immune responses to new peptides as shown in FIGS. 4 and 6, (may be partially 3). The immune responses to older cyclic peptide of FIG. 8, for S3B was considered to originated from earlier infection likely with old H3 virus.

It is further realized that the cyclic peptide 3 from H1 RPKVRDQ, FIG. 5, and corresponding sequences of current H3 RPRVRNI, and even to certain level older H3 sequence (now infecting more animals especially pigs) RPWVRGL, tested are substantially homologous with avian influenza H5 peptide 3 with sequence RPKVNGQ. It is thus realized that the peptides have tendency for conservation, especially H1 peptides are preferred because of conservation from spanish flu ((A(South Caroline/1/18). The invention is in a preferred embodiment directed to use of the preferred peptides 2 and 3, more preferably

The novel H1 and H3 peptides 2 and 3 showed strong immune reactions especially in persond who had been indicated to have been infected recently with influenza. The invention also revealed that linear peptide 3 of current H3 influenza comprising a conformational additional amino acid residue(s) including proline at the carboxyl terminus was especially effective in binding with certain antibodies.

Experimental Process

Materials and Equipments

Plates:
Reacti-Bind Streptavidin Coated Clear Strip Plates with Blocker BSA, Pierce, prod. no 15121

Reagents:
PBS, Phosphate Buffered Saline, 10 mM Na-phosphate buffer, 0.15 M NaCl, pH 7.2
Washing buffer: 0.2% BSA in PBS with 0.05% Tween-20.
BSA, Bovine Serum Albumin

Equipments:
Certomat RM, B. Braun Biotech International
Multiscan Spectrum (re w cuvette), Thermo Electron

Procedure:

Blocking: Incubation with 150 μl of 0.5% BSA in PBS with 0.05% Tween-20 for 1 h at room temperature (RT) with shaking (75 rpm, Certomat).

Washing: Three times with 200 μl of PBS with 0.05% Tween-20 with shaking for three minutes (150 rpm, Certomat).

Antigen binding: Incubation with 100 pmol of biotinylated peptide in 100 μl PBS for 0.5 h at RT with shaking (75 rpm, Certomat) and then overnight at +4° C.

Washing: Each well five times with 200 μl of Washing buffer, incubation each time for three minutes with shaking (150 rpm, Certomat).

Primary antibody: Serum from eight individuals were used as primary antibody dilutions, the serial dilutions (in Washing buffer) were: 1:10, 1:100, and 1:1000.

Incubation with 100 μl of diluted serum for 1 h at RT with shaking (75 rpm, Certomat). Washing: Ten times with 200 μl of Washing buffer, incubation each time for three minutes with shaking (150 rpm, Certomat).

Enzyme labeled secondary antibody:As secondary antibody 1:30000 dilution of Anti-Human Polyvalent Immunoglobulins (G, A, M) Peroxidase conjugate (Sigma) was used. Incubation with 100 μl of diluted immunoglobulins reagent for 1 h at RT with shaking (75 rpm, Certomat). Washing: Eight times with 200 μl of Washing buffer, incubation each time for three minutes with shaking (150 rpm, Certomat).

Determining binding activity: Incubation with 100 μl of TMB+ Substrate Chromogen (S5199, DacoCytomation, CA, USA) for 15 minutes at RT with shaking (75 rpm, Certomat).

Ending the enzymatic reaction by 100 μl 1 M H₂SO₄, shaking (75 rpm, Certomat) for three minutes. Measuring the absorbance at 450 nm.

Serum dilutions without antigen (=biotinylated peptide) were measured for unspecific binding (i.e. control samples).

Peptides 1B-5B

(Aminocaproyl=aminohexanoyl, biotin at N-terminus) H=hemagglutinin

Peptide 1B. Biotin-aminocaproyl-GTSSACIRR and represents the peptide 2 from current H3 variant

Peptide 2B. Biotin-aminocaproyl-SRPRVRNIP and represents the peptide 3 from current H3 variant

Peptide 3B. Biotin-aminocaproyl-CRPKVRDQC, cyclic peptide having disulfide bridge from Cys to Cys and represents the peptide 3 from former H1 variant

Peptide 4B. Biotin-aminocaproyl-CRPWVRGVC, cyclic peptide having disulfide bridge from Cys to Cys and represents the peptide 2 from former H3 variant; similar to Peptide 3 except that this is cyclic

Peptide 5B. Biotin-aminocaproyl-GVSASCSH and represents the peptide 2 from H1 variant

Serum Indications

Serum 1B (S1B). Individual indicates that according to symptoms he/she most probably had influenza on spring 2007. Serum of this individual was studied on ELISA experiments performed 2006, serum number was S2 (in Example 2).

Serum 2B (S2B). No indication of influenza. Serum of this individual was studied on ELISA experiments performed 2006, serum number was S5.

Serum 3B (S3B). Diagnosis made by medical doctor indicates that individual had influenza on spring 2007. Symptoms were so severe that he/she was hospitalized for one day. Has had also influenza on 1999.

Serum 4B (S4B). No indication of influenza. Serum of this individual was studied on ELISA experiments performed 2006, serum number was S6.

Serum 5B (S5B) Individual has been vaccinated against influenza on Winter 2002-2003 at USA.

Serum 6B (S6B). No indication of influenza. Serum of this individual was studied on ELISA experiments performed 2006, serum number was S4.

Serum 7B (S7B). Individual indicates that he/she had influenza on spring 1997. Serum of this individual was studied on ELISA experiments performed 2006, serum number was S3.

Serum 8B (S8B). No indication of influenza for this individual.

### Example 6

Blast (enterez web site) searches were performed with amino acid sequences Peptides 1-3. Similarity in human genome sequences were found especially for peptide 1 of H1 and H3. Relevance of the simirality is analyzed by estimating presence of the structures on cell surface proteins and on proteins surfaces when/if 3D structures are available. Three dimensional structures on patients (human or animal) peptides are considered.

### Example 7

Polyvalent conjugates of Peptide 1, Peptide 2 and Peptide 3 spacer modified (amihenoyl spacer) KLH protein are produced. Mice are immunized with conjugates and specific immune responses are observed. The example indicates suitability of the peptides for animal immunization. Similar experiments are performed with preferred animal patients: pigs and chicken to which the human viruses are more relevant and with horses. The human antibody data indicates as retrospective clinical trial usefulness for specific treatment of human. It is realized that immunization can be performed in multiple ways cited in the references of the application.

### Example 8

Further analysis of human immune responses to cyclic and linear peptides.

Material and Methods:

The analysis of antibody reactivity with serum of persons who had influenza was performed as in EXAMPLE 4. The test person and peptides 2B and 3B were same as in EXAMPLE 3. Serum of test subject S3B was frozen and the same serum as in EXAMPLE4, with other test subjects fresh serum was taken and may include further antibodies from recent contact with influenza virus. Test subjects 2B and 5B had respiratory infection in February 2008 lasting at least one week and involving high fever.

Peptides 1C-3C

(Aminocaproyl=aminohexanoyl, biotin at N-terminus) H=hemagglutinin

Peptide 1C. Biotin-aminocaproyl-CRPRVRNICG-NH4 (glycine amide at C-terminus), cyclic peptide having disulfide bridge from Cys to Cys and represents the peptide 3 from former H3 variant; Peptide 3 in cyclic form

Peptide 2B. Biotin-aminocaproyl-SRPRVRNIP and represents the peptide 3 from current H3 variant in linear form, same as in EXAMPLE 4. This is used especially for comparison to 1C (minimum sequence RPRVRNI)

Peptide 2C. Biotin-aminocaproyl-CRSKVNGQCG-NH4 (glycine amide at C-terminus), cyclic peptide having disulfide bridge from Cys to Cys and represents the peptide 3 from hemagglutinin of avian influenza/human infecting avian influenza with pandemic risk.

Peptide 3C. Biotin-aminocaproyl-RPKVRDQ, linear peptide 3 from hemagglutinin H1 common variant

Peptide 3B. Biotin-aminocaproyl-CRPKVRDQC, cyclic peptide having disulfide bridge from Cys to Cys and represents the peptide 3 from H1 hemagglutinin variant, Peptide 3 in cyclic form for comparision to linear H1 peptide 3C, same as in EXAMPLE 4.

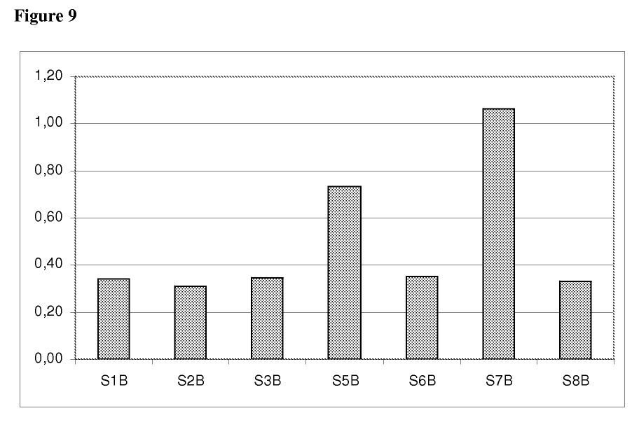

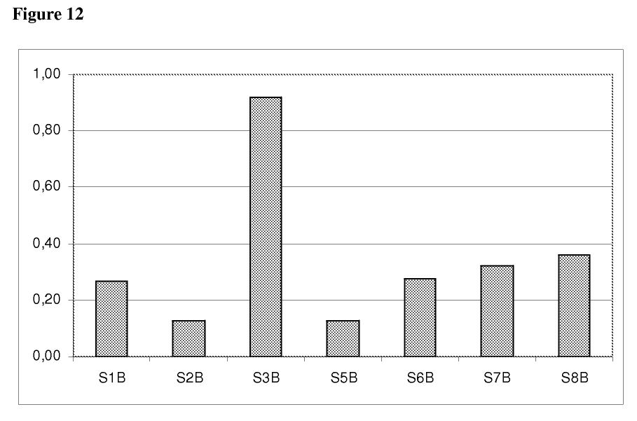

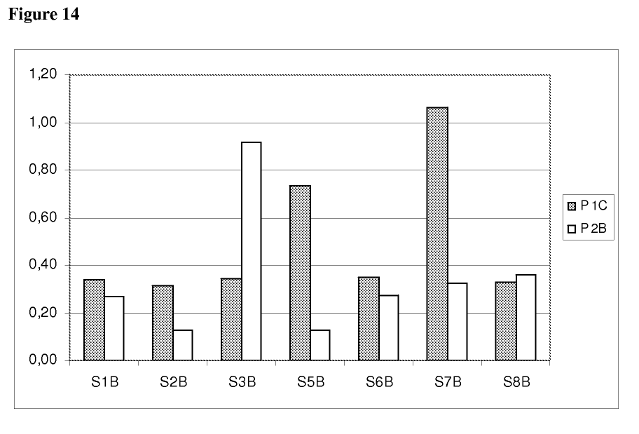

Results. FIG. 9 show reactivity with recent H3 influenza virus peptide 3 in cyclic form, peptide 1C, with sera of test subjects. Strong responses were observed from the serum of test subjects S5B an S7B. FIG. 12 shows corresponding and much weaker reactions to the linear corresponding peptide 2B, interestingly S3B had high reaction with linear peptide but weaker reaction with cyclic peptide indicating potential immunization but weaker activity of antibodies reacting to peptide conformation on virus surface. The FIG. 14 shows the comparision of the cyclic and linear peptide. The cyclic peptide was more reactive with all subjects except S3B, who likely has T-cell receptor based immunity not so useful for the 3D epitopes. The stronger reactivity against cyclic peptide in subjects S7B, S5B and even S2B indicates specific immune reaction against the conformation of the cyclic peptide epitope on virus surface.

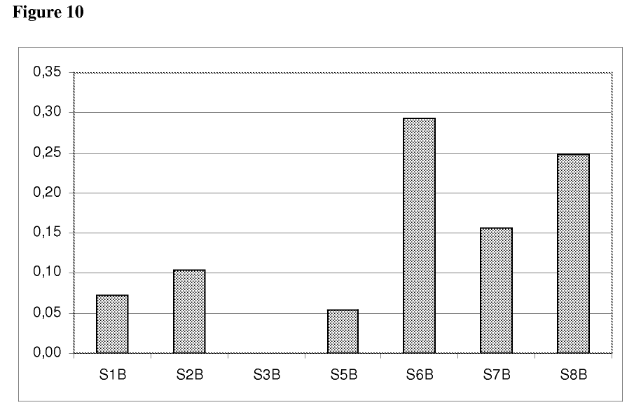

FIG. 10 shows serum reactions to avian influenza virus H5 peptides. Strongest reactivities was anticipated have some correlation of being contact with chicken (under farm conditions) in childhood by test subjects S2B, S6B, and S8B, which may have included immunization to avian influenza hemagglutinin. It is realized that there are natural antibodies against the key 3D cyclic peptide epitope of H5 influenza virus, which has potential for the analysis of the infections and/or presence of protecting antibodies, and/or search/selection of antibodies and these H5 hemagglutinin structures are also preferred for vaccination against influenza.

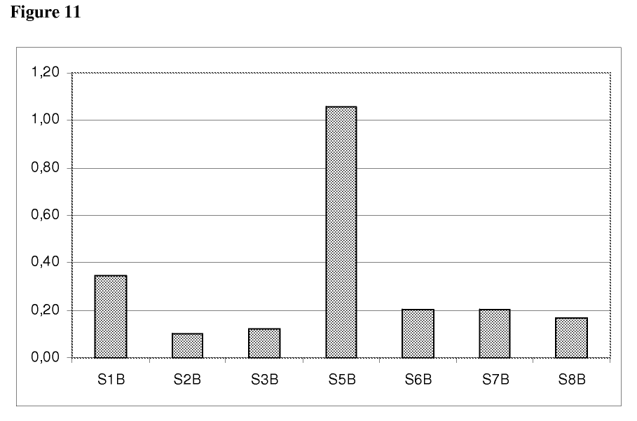

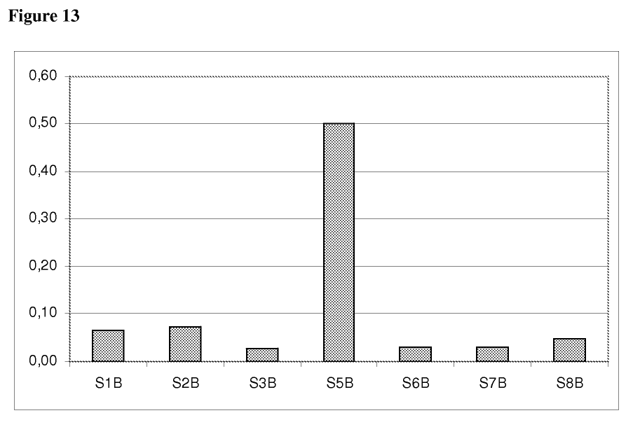

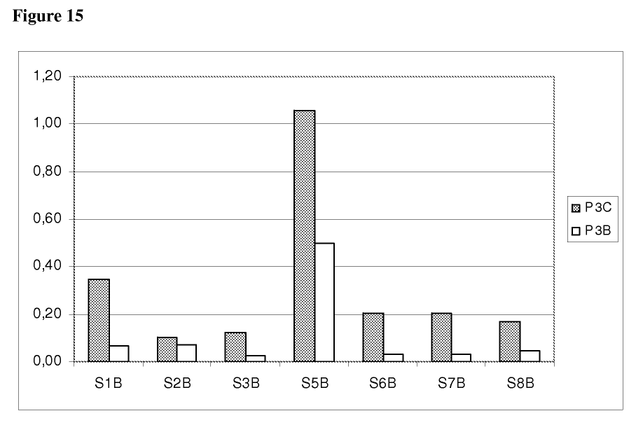

FIG. 11 shows reactivity with recent H1 influenza virus peptide 3 in linear form, peptide 3C, with sera of test subjects. Strong responses were observed from the serum of test subjects S5B. FIG. 13 shows corresponding and reactions to the linear corresponding cyclic peptide 3B, interestingly S5B had higher reaction with linear peptide but weaker reaction with cyclic peptide indicating potential immunization but somewhat weaker but still substantial activity of antibodies reacting to peptide conformation on virus surface. The immunization mechanism has been likely a combination of T-cell and conformational (B-cell/antibody) responses. The FIG. 15 shows the comparison of the cyclic and linear peptide. The linear peptide was more reactive with low signals with other subjects, indicating likely no contact or immunization with H1 hemagglutinin 3D epitopes by the subjects, the immune response of S5B has been likely derived from vaccination.

TABLE 1
Approximate immune reactions of sera from test subjects
1-6 against synthetic peptides.

| | P1-N | P1-C Cys | P2-N | P2-mid Cys | P3-N | HA11-N Cys |
|---|---|---|---|---|---|---|
| Serum 1 | − | − | ++ | + | ++ | + |
| Serum 2 | + | + | ++++ | +++ | + | +++ |
| Serum 3 | ++ | ++ | ++ | +++ | − | ++ |
| Serum 4 | ++ | ++ | +++ | + | − | ++ |
| Serum 5 | − | − | +++ | + | + | + |
| Serum 6 | − | +++ | +++ | +++ | + | ++ |

P1, P2, And P3 indicates peptides 1-3,
HA11 is commercial peptide
N is N-terminal Biotin immobilized conjugate,
Cys-indicates Cys-conjugate,
C is C-terminal.

1.-33. (canceled)

34. Peptide conjugate according to Formula
[PEP-(y)ₚ-(S)_{q}-(z)ᵣ-]ₙPO
wherein PEP is a peptide epitope;
n is an integer ≧1 indicating the number of PEP groups covalently attached to carrier PO; S is a spacer group;
y and z are linking groups so that at least y or z is a linking atom group;
p, q and r are independently 0 or 1 so that at least p or r is 1;
PO is an oligomeric or polymeric carrier structure;
wherein said PEP is a cyclic peptide comprising a 7-mer peptide derived from H1, H3, or H5 hemagglutinin of influenza virus, said peptide having a sequence the location of which in said hemagglutinin corresponds to the loop sequence at positions 220-226 of X31-hemagglutinin.

35. The conjugate according to claim 34, wherein said 7-mer peptide has a loop structure conformationally similar to the loop sequence at positions 220-226 of X31-hemagglutinin.

36. The conjugate according to claim 34, wherein said cyclic peptide comprises the H1, H3 and H5 concensus sequence according to Formula
(C)ₙRX₁X₂VX₃ (SEQ ID NO:1), wherein
X₁ is P or S,
X₂ is R or K, and
X₃ is R or N,
(C) is optional cysteine of the cyclic peptide and n is 0 or 1 indicating its presence or absence or
the cyclic peptide comprises H3 type sequence

| | |
|---|---|
| (C)ₙRPRVR | (SEQ ID NO: 12) |

or
H1 and H5 sequences with consensus according to Formula

| | |
|---|---|
| (C)ₙRX₁KV, wherein | (SEQ ID NO: 13) |

X₁ is P or S, and
(C) is optional cysteine of the cyclic peptide and n is 0 or 1 indicating its presence or absence.

37. The conjugate according to claim 34, wherein the sequence of said cyclic peptide comprises H1 type sequence KVR, H3 type sequence RVR or H5 type sequence KVN.

38. The conjugate according to claim 34, wherein the sequence of said cyclic peptide comprises H1 type sequence KVR, H3 type sequence RVR or H5 type sequence KVN with cysteines added to both ends of the 7-mer peptide resulting in the following structures:

| | |
|---|---|
| CXXKVRXXC, | (SEQ ID NO: 2) |
| CXXRVRXXC, | (SEQ ID NO: 3) |
| or | |
| CXXKVNXXC; | (SEQ ID NO: 4) |

wherein X is any amino acid derived from H1, H3, or H5 hemagglutinin of influenza virus.

39. The conjugate according to claim 34, wherein the sequence of said cyclic peptide comprises RPRVRNI (SEQ ID NO:5), RPRIRNI (SEQ ID NO:6), RSKVNGQ (SEQ ID NO:7), or RPKVRDQ (SEQ ID NO:8).

40. The conjugate according to claim 38, wherein the carboxyl terminal cysteine is further linked to a spacer, additional hemagglutinin peptide or glycine amide.

41. The conjugate according to claim 34, wherein z is selected form the group: a chemoselective ligation group or biotin or equivalent ligand capable of specific strong non-covalent interaction.

42. The conjugate according to claim 34, wherein PO is selected from the group consisting of: solid phases, immunogenic and/or oligomeric or polymeric carrier such as multiple antigen presenting (MAP) constructs, proteins such as KLH (keyhole limpet hemocyanin), and oligosaccharide or polysaccharide structures.

43. The conjugate according to claim 34, wherein said z or y is selected from a group consisting of: a linking atom group formed from sulphur atom of a cysteine residue, preferably linked to maleimide or analogous structure or to a sulphur of cysteine in the matrix or the linking group is a strong non-covalent interaction formed by binding of a ligand to a protein, preferably biotin binding to an avidin protein, and an O-hydroxylamine residue —O—NH— or —O—N═, with the nitrogen atom being linked to the OS or PO structure, respectively.

44. Cyclic peptide of 7-12 amino acids comprising the sequence selected from the group consisting of RPRVRNI (SEQ ID NO:5), RPRIRNI (SEQ ID NO:6), or RSKVNGQ (SEQ ID NO:7), CRPRVRNIC (SEQ ID NO:9), CRPRIRNIC (SEQ ID NO:10), or CRSKVNGQC (SEQ ID NO:11); or any of the peptides ID NO 9-11 or cyclic peptide CRPKVRDQC (SEQ ID NO:14), wherein the carboxyl terminal cysteine of said peptide is further linked to a spacer, additional hemagglutinin peptide or glycine amide.

45. A method for evaluating the potential of a chemical entity to bind to the conjugate according to claim 34 comprising the steps of:
(i) contacting said chemical entity with said conjugate or peptide under conditions that allow said chemical entity to bind said conjugate or peptide; and
(ii) detecting the presence of a complex of said chemical entity and said conjugate or peptide.

46. The method according to claim 45, wherein said chemical entity is an antibody.

47. The method according to claim 45, wherein the use of the method is selected from the group consisting of: vaccine development, selection of antibodies from a library of antibodies; an ex vivo or in vivo immunization method, screening binding agents from a library; screening antibodies, wherein said chemical entity is an antibody and said peptide is contacted with a sample of whole blood, plasma or serum, an in vitro immunoassay or in vitro selection of an antibody library such as phage display antibody library, a method of detecting the presence of an antibody in a biological sample.

48. The method according to claim 47, wherein the use of the method is producing a peptide vaccine against influenza, the method further comprising steps of:
administering the conjugate according to claim 34 to an animal; and
monitoring the animal in order to detect immune response against the conjugate.

49. The conjugate according to claim 34 as a part of a vaccine composition.

50. The conjugate according to claim 49 further comprising a linear peptide having the same sequence as the cyclic peptide.

51. The method according to claim 45 for identifying influenza virus in a biological sample, the method further comprising:
(a) contacting the biological sample with an antibody substance capable of binding the conjugate according to claim 34; and
(b) detecting the binding between said antibody substance and influenza virus or part thereof in the sample, said binding indicating the presence and type of influenza virus in the sample.

52. The method according to claim 45 for screening variants of a 7-mer peptide derived from H1, H3, or H5 hemagglutinin of influenza virus, said peptide having a sequence the location of which in said hemagglutinin corresponds to the loop sequence at positions 220-226 of X31-hemagglutinin; the method comprising the steps of:
(a) acquiring sequence data of hemagglutinin from data banks or by sequencing influenza virus genomes;
(b) obtaining candidate peptide sequences by comparing the sequences to known sequences from the same location;
(c) preparing a cyclic peptide or a conjugate as defined in claim 34;
(d) screening antibodies binding to the peptide or conjugate obtained in step (c).

53. The method according to claim 52, wherein the method further involves a step of search of any of the peptide epitopes 1-3, more preferably the peptide 3, of an hemagglutinin from database comprising human genome coded peptide sequences and selection of peptides, which are not expected to cause immune reaction against the human (or animal) subject.

54. A method for evaluating the potential of a chemical entity to bind to the cyclic peptide according to claim 44 comprising the steps of:
(i) contacting said chemical entity with said conjugate or peptide under conditions that allow said chemical entity to bind said conjugate or peptide; and
(ii) detecting the presence of a complex of said chemical entity and said conjugate or peptide.

55. The method according to claim 45 for identifying influenza virus in a biological sample, the method further comprising:
(a) contacting the biological sample with an antibody substance capable of binding the cyclic epitope according to claim 44; and
(b) detecting the binding between said antibody substance and influenza virus or part thereof in the sample, said binding indicating the presence and type of influenza virus in the sample.


SEQUENCE LISTING (14 sequences)

SEQ ID NO: 1 - protein, 6 aa, Influenza virus
  MISC_FEATURE (1)..(1): Xaa is Cys or nothing
XRXXVX

SEQ ID NO: 2 - protein, 9 aa, Influenza virus
  MISC_FEATURE (2)..(2): Xaa is any amino acid
CXXKVRXXC

SEQ ID NO: 3 - protein, 9 aa, Influenza virus
  MISC_FEATURE (2)..(2): Xaa is any amino acid
CXXRVRXXC

SEQ ID NO: 4 - protein, 9 aa, Influenza virus
  MISC_FEATURE (2)..(2): Xa is any amino acid
CXXKVNXXC

SEQ ID NO: 5 - protein, 7 aa, Influenza virus
RPRVRNI

SEQ ID NO: 6 - protein, 7 aa, Influenza virus
RPRIRNI

SEQ ID NO: 7 - protein, 7 aa, Influenza virus
RSKVNGQ

SEQ ID NO: 8 - protein, 7 aa, Influenza virus
RPKVRDQ

SEQ ID NO: 9 - protein, 9 aa, Influenza virus
CRPRVRNIC

SEQ ID NO: 10 - protein, 9 aa, Influenza virus
CRPRIRNIC

SEQ ID NO: 11 - protein, 9 aa, Influenza virus
CRSKVNGQC

SEQ ID NO: 12 - protein, 6 aa, Influenza virus
  MISC_FEATURE (1)..(1): Xaa is Cys or nothing
XRPRVR

SEQ ID NO: 13 - protein, 5 aa, Influenza virus
  MISC_FEATURE (3)..(3): Xaa is Pro or Ser
CRXKV

SEQ ID NO: 14 - protein, 9 aa, Influenza virus
CRPKVRDQC